SEQUENCE LISTING (12 sequences)

SEQ ID NO: 1 - protein, 70 aa, Plasmodium berghei
GYGQNKSIQAQRNLNELCYNEGNDNKLYHVLNSKNGKIYIRNTVNRLLADAPEGKKNEKKNKIERNNKLK

SEQ ID NO: 2 - protein, 69 aa, Plasmodium berghei
NDDSYIPSAEKILEFVKQIRDSITEEWSQCNVTCGSGIRVRKRKGSNKKAEDLTLEDIDTEICKMDKCS

SEQ ID NO: 3 - protein, 16 aa, Artificial
  Representing Plasmodium berghei
GYGQNKSIQAQRNLNE

SEQ ID NO: 4 - protein, 16 aa, Artificial
  Representing Plasmodium berghei
RNLNELCYNEGNDNKL

SEQ ID NO: 5 - protein, 16 aa, Artificial
  Representing Plasmodium berghei
NDNKLYHVLNSKNGKI

SEQ ID NO: 6 - protein, 16 aa, Artificial
  Representing Plasmodium berghei
KNGKIYIRNTVNRLLA

SEQ ID NO: 7 - protein, 16 aa, Artificial
  Representing Plasmodium berghei
NRLLADAPEGKKNEKK

SEQ ID NO: 8 - protein, 15 aa, Artificial
  Representing Plasmodium berghei
KNEKKNKIERNNKLK

SEQ ID NO: 9 - protein, 19 aa, Artificial
  Representing Plasmodium berghei
NDDSYIPSAEKILEFVKQI

SEQ ID NO: 10 - protein, 19 aa, Artificial
  Representing Plasmodium berghei
FVKQIRDSITEEWSQCNVT

SEQ ID NO: 11 - protein, 25 aa, Artificial
  Representing Plasmodium berghei
QCNVTCGSGIRVRKRKGSNKKAEDL

SEQ ID NO: 12 - protein, 18 aa, Artificial
  Representing Plasmodium berghei
KKAEDLTLEDIDTEICKM

(19) United States
(12) Patent Application Publication
(10) Pub. No.: US 2009/0092619 A1
(43) Pub. Date: Apr. 9, 2009

(54) METHODS AND COMPOSITIONS FOR MALARIA PROPHYLAXIS

(76) Inventors: Photini Sinnis, New York, NY (US); Alida Coppi, Flushing, NY (US); Elizabeth Nardin, Leonia, NJ (US)

Correspondence Address: KOHN & ASSOCIATES, PLLC, 30500 NORTHWESTERN HWY, SUITE 410, FARMINGTON HILLS, MI, 48334, US

(21) Appl. No.: 11/866,738

(22) Filed: Oct. 3, 2007

Related U.S. Application Data
(63) Continuation-in-part of application No. 11/200,723, filed on Aug. 10, 2005.
(60) Provisional application No. 60/600,547, filed on Aug. 11, 2004.

(51) Int. Cl. A61K 39/395; C07K 16/00

(52) U.S. Cl. 424/141.1; 530/387.1; 530/388.1; 424/130.1

(57) ABSTRACT

A composition for preventing malaria infection including a steric inhibitor of circumsporozoite protein cleavage. A pharmaceutical composition for preventing malaria infection including a steric inhibitor and a pharmaceutical carrier. A method of malaria infection prophylaxis including the step of administering an effective amount of the composition of the present invention. A method of malaria prophylaxis by sterically inhibiting circumsporozoite protein processing or by directly inhibiting a protease of a sporozoite from binding to its target. Methods of preventing sporozoite cell invasion or preventing circumsporozoite processing through steric or direct inhibition.

### CROSS REFERENCE TO RELATED APPLICATIONS

This application is a Continuation-in-Part of U.S. patent application Ser. No. 11/200,723, filed Aug. 10, 2005, which claims benefit under 35 U.S.C. Section 119(e) of U.S. Provisional Patent Application No. 60/600,547, filed Aug. 11, 2004, both of which are incorporated herein by reference in their entirety.

### GOVERNMENT SUPPORT

Research in this application was supported in part by contracts from National Institute of Health (R01 AI044470, R01 AI056840, R01 AI025085). The government has certain rights in the invention.

### BACKGROUND OF THE INVENTION

1. Technical Field

The present invention relates to compositions and methods for prophylaxis and treatment of malaria infection.

2. Background Art

Malaria is a devastating infectious disease. There are over 300 million cases per year worldwide and it is responsible for over one million deaths per year. Malaria is caused by protozoan parasites of the genus Plasmodium. There are four species that infect humans and they are all transmitted by the bite of an infected Anopheline mosquito. Plasmodium falciparum is responsible for most of the death due to malaria; however, Plasmodium vivax is the most prevalent species worldwide and causes a significant amount of morbidity. Plasmodium falciparum, the cause of the most virulent form of malaria, has developed resistance to currently used drugs. This in turn has led to an increase in the incidence of malaria and to fewer drugs for both treatment and prophylaxis of the disease.

Malaria infection is initiated when an infected Anopheline mosquito injects sporozoites into a subject during the mosquito's blood meal. After injection, the parasite enters the bloodstream and undergoes a series of changes as part of its life-cycle. The sporozoite travels to the liver where it invades hepatocytes. One sporozoite can generate over 10,000 hepatic merozoites, which will then rupture from the hepatocyte and invade erythrocytes.

The sporozoite stage of Plasmodium is unique in that it is invasive twice in its lifetime: In the mosquito, sporozoites emerge from oocysts on the midgut wall and invade salivary glands. Then as the mosquito probes for blood, they are injected into the dermis of the mammalian host where they wait to be injected into a mammalian host as the mosquito probes for blood. In the mammalian host further development of the parasite requires that sporozoites invade hepatocytes and develop into exoerythrocytic stages (EEFs). Previous studies have shown that sporozoites can interact with cells in one of two ways: they can productively invade a cell, forming a parasitophorous vacuole in which they will replicate, or they can migrate through a cell, breaching the cell's plasma membrane in the process (Mota et al., 2001). The ability to traverse cell barriers likely enables sporozoites to reach the liver from their injection site in the dermis.

The invasive zoites of Apicomplexan protests share a highly conserved structural organization that confers a similar overall mechanism to target cell invasion. Cell invasion by Plasmodium is an ordered process in which the parasite forms a close association with the host cell plasma membrane and then actively enters the cell (Sinnis, et al. (1997)). This process is aided by the sequential and regulated secretion of proteins from apical organelles called micronemes and rhoptries (Carruthers, et al. (1999)). Many of these secreted proteins contain adhesive domains and studies with protease inhibitors demonstrate that proteolytic cleavage of both zoite surface adhesions as well as adhesions secreted from apical organelles is required for invasion (reviewed in Carruthers and Blackman, 2005). Frequently these proteins undergo complex processing reactions that include both NH₂ and COOH-terminal cleavage. Although the function of these cleavage events has not been definitively demonstrated, it has been suggested that removal of a NH₂-terminal “prodomain” may expose cell-adhesive motifs or change the conformation of the protein such that these cell-adhesive motifs can now adhere to their target cell receptors and COOH-terminal cleavage leads to the release of the adhesin, possibly in association with its host cell receptor, from the zoite surface, enabling forward movement into the target cell (Carruthers and Blackman, 2005).

One of these secreted proteins is the circumsporozoite protein (hereinafter, “CSP”). Studies have shown that CSP mediates sporozoite adhesion to target cells (Sinnis, et al. (2002)) and that it is required for sporozoite development in the mosquito (Menard, et al. (1997)). CSP forms a dense coat on the sporozoite and is constitutively secreted onto the parasite's surface. However, secretion of the protease that cleaves CSP appears to be regulated since there is a dramatic increase in the kinetics of CSP cleavage when sporozoites are added to cells. In the absence of cells, it takes two hours for newly synthesized CSP to be cleaved. In contrast, within minutes of contacting target cells, the majority of sporozoites no longer have full-length CSP on their surface (Coppi et al., 2005).

A similar phenomenon occurs in the merozoite stage of Plasmodium where a low level of MSP-1 cleavage is observed in the absence of cells. During invasion, however, processing goes to completion within minutes (Blackman, et al. (1990) and Blackman, et al. (1993)). It is likely that low-level cleavage in the absence of cells is due to leaky secretion from apical organelles whereas exocytosis of larger amounts of protease is mediated by specific signals that are transduced upon cell contact.

The Plasmodium proteins that are proteolytically processed during cell invasion can be divided into 2 groups: those that are secreted onto the parasite surface during invasion (e.g., TRAP and AMA-1) and those that are already on the surface (e.g., CSP and MSP-1), which are the major surface proteins of sporozoites and merozoites respectively. In neither case is the precise function of cleavage in the invasion process known. However, it is noteworthy that in the case of the surface proteins, CSP and MSP-1, the C-terminal fragment remaining with the parasite contains a known cell adhesive motif. Proteolytic cleavage may control exposure of these cell-adhesive motifs.

Previous work has demonstrated that proteolytic cleavage of Plasmodium proteins during invasion is accomplished primarily by serine proteases. CSP, however, is cleaved by a cysteine protease. In addition, a recent study found that the cysteine protease falcipain-1 is required for merozoite invasion of erythrocytes (Greenbaum, et al. (2002)). Thus, in addition to serine proteases, cysteine proteases are important components of Plasmodium's invasion machinery.

More specifically referring to the CSPs, they all contain a central repeat region whose amino acid sequence is species-specific. Immediately before the repeat region is a highly conserved five amino acid sequence called region I and in the C-terminal portion of CSP is a known cell adhesive sequence with similarity to the type I thrombospondin repeats (hereinafter, “TSR”)(Goundis, et al. (1988)). CSP has a canonical glycosylphosphatidyl inositol (hereinafter, “GPI”) anchor addition sequence in its C-terminus (Moran, et al. (1994)) and the CSP is GPI-anchored to the sporozoite plasma membrane. CSP immunoprecipitated from metabolically-labeled sporozoites consists of 1 to 2 high MW bands (that differ by ˜1 kDa) and a low MW band that is 8 to 10 kDa smaller (Yoshida, et al. (1981); Cochrane, et al. (1982); Krettli, et al. (1988); and Boulanger, et al. (1988)). Biosynthetic studies show that an initial label is incorporated into the top band(s) and the lower MW band appears only later as a processed product (Yoshida, et al. (1981) and Cochrane, et al. (1982)). Recent studies from Applicants' laboratory have shown that CSP is proteolytically cleaved by a papain-family cysteine protease and the entire N-terminal third of the CSP is removed (Coppi et al., 2005).

Applicants have recently found that CSP is proteolytically processed by a parasite cysteine protease during invasion of hepatocytes (Coppi et al., 2005). Processing occurs extracellularly, on the sporozoite surface and results in the removal of the NH₂-terminal third of the protein. CSP cleavage occurs when sporozoites contact hepatocytes and the cysteine protease inhibitor, E-64, inhibits CSP processing and abolishes sporozoite infectivity in the mammalian host. Although this data suggests that proteolytic processing of CSP is required for sporozoite entry into hepatocytes, the broad substrate specificity of E-64 did not allow for determination of whether CSP cleavage was specifically required for sporozoite infectivity. In addition, previous studies were unable to determine the precise cleavage site within the NH₂-terminal portion of CSP.

All of the symptoms of malaria are associated with the erythrocytic stage of the disease and treatment of malaria infection requires targeting this stage. The anti-malarial drugs currently on the market target the erythrocytic stage of the parasite. Unlike Plasmodium falciparum, in most parts of the world, Plasmodium vivax is still sensitive to chloroquine. However, in Plasmodium vivax malaria, treatment of the erythrocytic stages is not adequate for eradicating the infection because this parasite has dormant liver stages that can cause relapses months to years after the blood infection has been cleared. Plasmodium vivax-infected individuals must also take primaquine, the only drug that is effective against liver stages of the disease. Primaquine is contraindicated in people with glucose-6 phosphate dehydrogenase deficiency and in pregnant women. Thus, at least two drugs must be taken to prevent Plasmodium vivax infection.

Currently, there are no previously described drugs that target the sporozoite stage of the parasite. The advantages of targeting this stage of the parasite include, but are not limited to, preventing malaria infection in travelers or military personnel going into endemic areas, no longer requiring treatment of P. vivax infections with primaquine, and slowing the development of drug resistance because it targets a stage of the parasite that does not multiply and that uses very low numbers to establish infection.

The incidence of malaria is increasing owing to several factors including resistance of the parasite to currently available anti-malarial drugs. In addition, efforts to develop an effective malaria vaccine have not been successful. Therefore, there is an urgent need to identify new parasite drug targets both for prophylaxis and therapy. Potential new targets include Plasmodium proteases due to their critical roles in the parasite life cycle and the feasibility of developing specific inhibitors.

Current research in both Plasmodium and other Apicomplexan parasites such as Toxoplasma demonstrates that proteolytic cleavage of parasite surface and secreted proteins is necessary for successful invasion of host cells (Blackman, M. J., Howell, S. A., et al., and Kim, K.). It has been recently shown that the major surface protein of sporozoites, the circumsporozoite protein (CSP), is proteolytically processed by a parasite-derived cysteine protease (Coppi et al., 2005). This cleavage event is temporally associated with sporozoite invasion of hepatocytes.

One particular cysteine protease inhibitor is allicin. Allicin is one of the active compounds of freshly crushed garlic that has been shown to possess a number of antimicrobial activities (Ankri, S. and Harris, L. C.). Allicin has a broad spectrum of antibacterial effects, demonstrating activity against Gram-positive, Gram-negative, and even acid-fact bacteria (Uchida, Y.). Antifungal properties of allicin have been observed not only in vitro, but also recently in vivo (Shadkchan, Y.).

Less work has been done to elucidate the effect of allicin on parasitic protozoa. Allicin inhibits the growth of various parasitic protozoa and extracts of allicin have been effective against a host of infections, including Giardia, Leishmania, and Trichomonas (Reute, H. D.). Because of its sulfydryl modifying activity (Willis, E.), the effects of allicin are thought to involve the inhibition of thiol-containing enzymes in microorganisms (Rabinkov, A.). In fact, allicin has been shown to irreversibly inhibit papain (Rabinkov, A.). Allicin rapidly penetrates cell membranes (Miron, T.), allowing it to quickly exert its biological effects. In the parasitic protozoan Entamoeba histolytica, allicin inhibits cysteine proteases of the parasite, inhibiting parasite growth (Mirelman, D.) and preventing its cytopathic effects (Ankri, S.). Recently, Applicants showed that allicin inhibited the cysteine protease responsible for CSP cleavage and thereby inhibited sporozoite infectivity (Coppi et al., 2006).

Accordingly, there is a need for a composition and related methods for the prophylaxis and treatment of malaria infection whereby proteolytic cleavage of sporozoites' CSP is prevented, which results in inhibiting cell entry of the sporozoites. Thus, malaria infection is prevented or aborted.

Herein Applicants use a genetic approach and show that the cleavage site of CSP is located in the highly conserved 5 amino acid sequence called region I and that CSP cleavage is required for efficient invasion of hepatocytes by sporozoites. In addition, it is shown that sporozoite-neutralizing antibodies sterically block CSP processing. Together, these data raise the possibility that inhibition of CSP cleavage can be a viable approach to targeting the preerythrocytic stages of Plasmodium.

### SUMMARY OF THE INVENTION

The present invention provides a composition for preventing malaria infection including a steric or direct inhibitor. Further, the present invention provides a pharmaceutical composition for preventing malaria infection including an effective amount of an inhibitor that blocks association between the protease and its target, CSP as well as a pharmaceutical carrier. The present invention also provides a method of malaria infection prophylaxis including the step of administering an effective amount of the composition of the present invention. Additionally, the present invention provides a method of malaria prophylaxis by inhibiting circumsporozoite protein processing. Furthermore, the present invention provides a method of malaria prophylaxis by inhibiting a protease of a sporozoite. Finally, the present invention provides various methods of preventing sporozoite cell invasion or preventing circumsporozoite processing.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates that antiserum to the N-terminal portion of CSP does not recognize the low molecular weight form of circumsporozoite protein (CSP);

FIG. 2 illustrates that CSP processing is inhibited by cysteine and some serine protease inhibitors;

FIG. 3 illustrates that CSP is processed extracellularly by a parasite protease;

FIG. 4 illustrates E-64 inhibits sporozoite invasion of, but not attachment to, cells;

FIG. 5 illustrates processing of CSP is not required for sporozoite motility or migration through cells;

FIG. 6 illustrates E-64 inhibits sporozoite infectivity in vivo;

FIG. 7 illustrates that allicin prevents cleavage of CSP;

FIG. 8 illustrates that at low doses, allicin is not directly toxic to Plasmodium sporozoites;

FIG. 9 illustrates the effect of allicin on gliding motility;

FIG. 10 illustrates that allicin inhibits sporozoite invasion of host cells;

FIG. 11 illustrates that allicin decreases sporozoite infectivity in vivo;

FIGS. 12A-12D show the generation and verification of Recombinant Control sporozoites (hereinafter, “RCon”) and sporozoites in which region I has been deleted from CSP (hereinafter, “ΔRI”). FIG. 12A is a schematic of Plasmodium circumsporozoite protein (CSP) showing the central repeat region flanked by the highly conserved region I and type I thrombospondin repeat (TSR). The first 20 residues of CSP have the features of a eukaryotic signal sequence and the COOH-terminal sequence contains a canonical GPI-anchor addition site. Lines above the box show the length and location of the long peptides used to make antisera to the NH₂— and COOH— portions of CSP (Coppi et al., 2005). FIG. 12B shows the strategy used to replace the endogenous CSP locus with either wild type or region I-deleted CSP (giving rise to RCon and ΔRI sporozoites). The transfection plasmid (pCSRep) contains, from left to right, 730 bp of CSP 5′UTR (thin black line), the selectable marker hDHFR (black box) with its upstream and downstream control elements (thick black lines) and the CSP gene (black box with thin lines) flanked by its upstream and downstream control elements (thin black lines). The dotted grey lines indicate the location of homologous recombination with the endogenous locus (WT CSP Locus). Following this is a schematic of the locus after the desired recombination event. Thick dashed lines above the CSP gene show the area of homology with the probe used in the Southern blot and the size of the fragments in the endogenous and recombined loci expected to hybridize with the probe are shown below each respective locus. P1 and P2 show the positions of the primers used for diagnostic PCR and P3 and P4 show positions of the primers used to amplify and sequence CSP after recombination. FIG. 12C displays diagnostic PCR (left) and Southern blot (right). PCRs with primer pairs 1 & 2, and 3&4 whose locations are shown in (B). Primers 1 & 2 amplify an integration specific product in the recombinant control (RCon) and region I deletion (ΔRI) clones but not wild type untransfected parasites (WT). Primers 3 & 4 amplify the Pml-Pac CSP fragment which was sequenced to confirm the presence of the deletion (data not shown). Southern blot of EcoRV-digested genomic DNA probed with the Pml-Pac fragment of CSP showing the expected 4 kb band in WT parasites and a 7.4 kb band in RCon or ΔRI parasites. FIG. 12D displays phase-contrast and fluorescence images of RCon or ΔRI sporozoites fixed with paraformaldehyde and stained with mAb 3D11 directed against the repeat region of CSP. Bar=10 μm;

FIGS. 13A-13C show CSP processing in RCon and ΔRI parasites. FIG. 13A shows a Western blot of salivary gland RCon and ΔRI sporozoites probed with mAb 3D11 (left panel). As a loading control, blots were also probed with polyclonal antisera against TRAP (right panel). Molecular weight marker locations are shown to the left of each blot. FIG. 13B shows pulse-chase metabolic labeling of RCon and ΔRI salivary gland sporozoites. Sporozoites were metabolically labeled with ³⁵S-Cys/Met and kept on ice (time=0) or chased for the indicated times after which they were lysed, CSP was immunoprecipitated and analyzed by SDS-PAGE and autoradiography. FIG. 13C shows pulse-chase metabolic labeling of RCon and ΔRI salivary gland sporozoites in the presence of hepatocytes. Sporozoites were metabolically labeled with ³⁵S-Cys/Met and chased for 1 hr to give time for labeled CSP to be exported to the parasite surface. Sporozoites were then added to Hepa 1-6 cells for the indicated times after which parasites and cells were lysed, CSP was immunoprecipitated and analyzed by SDS-PAGE and autoradiography;

FIGS. 14A-14C show mosquito infection by RCon and ΔRI parasites. An. stephensi mosquitoes were allowed to feed on mice infected with erythrocytic stages of wild type (triangles), RCon (closed circles) or ΔRI (open diamonds) parasites. Mosquitoes were dissected on the indicated days post-feeding and the number of sporozoites associated with mosquito midguts (14A), hemolymph (14B) or salivary glands (14C) were determined. For each parasite line, 20 mosquitoes were harvested per time point and shown is the average number of sporozoites per mosquito. Midgut and hemolymph sporozoite counts were performed with 3 different batches of infected mosquitoes and shown are representative experiments. Salivary gland sporozoite counts were performed on 6 different batches of infected mosquitoes and shown is a representative experiment;

FIGS. 15A-15C show infectivity of RCon and ΔRI parasites in vitro. FIGS. 15A and 15B show attachment and invasion. Wild type, RCon and ΔRI sporozoites were pretreated with (black bars) or without (gray bars) E-64d and added to Hepa 1-6 cells for 1 hr. Cells were then fixed and stained using a double staining assay that distinguishes intracellular from extracellular sporozoites. Total number of attached sporozoites, which includes both intracellular and extracellular sporozoites, is shown in (A) and the percentage of these found intracellularly is shown in (B). 60 fields per well were counted, each point was performed in triplicate and shown are the means±standard deviations. The experiment was repeated twice with identical results. FIG. 15C shows liver stage development. Wild type, RCon and ΔRI sporozoites were added to Hepa 1-6 cells and after 48 hr cells were fixed, stained with mAb 2E6 and the number of EEFs were counted. At least 50 fields per well were counted, each point was performed in triplicate and shown are the means±standard deviations. The experiment was repeated twice with identical results;

FIGS. 16A and 16B show infectivity of RCon and ΔRI parasites in vivo. C57BI/6 (FIG. 16A) and Swiss Webster (FIG. 16B) mice were injected intravenously with 10⁴ sporozoites and 40 hr later, mice were sacrificed, total liver RNA was extracted and the parasite burden in the liver was measured by RT followed by quantitative PCR. Infection is expressed as the number of copies of P. berghei 18S rRNA. There are 5 mice per group and shown are the mean±standard deviations. This experiment was performed 3 times and shown is a representative experiment;

FIGS. 17A-17C show gliding motility and cell traversal by RCon and ΔRI sporozoites. FIGS. 17A and 17B show motility. Wild type, RCon and ΔRI sporozoites were added to wells for 1 hr at 37° C., fixed and stained with mAb 3D11 to visualize sporozoites and the trails of CSP shed during gliding motility. Shown in FIG. 17A is the percentage of sporozoites that exhibited gliding motility and in FIG. 17B is the number of these sporozoites associated with 1 (white bars), 2 to 10 (light gray bars), or >10 (black bars) circles per trail. Each point was performed in triplicate, 200 sporozoites/well were counted, and the means±standard deviations are shown. This experiment was performed 3 times and shown is a representative experiment. FIG. 17C shows migration. Hepa 1-6 cells were loaded with calcein green, the indicated sporozoite line was added to the cells for 1 hr at 37° C. and the fluorescent calcein-green released into the supernatant was measured. Each point was performed in triplicate and shown are the means±standard deviations. This experiment was performed 3 times and shown is a representative experiment;

FIGS. 18A and 18B show conformation of CSP before and after contact with hepatocytes. FIG. 18A shows phase-contrast and fluorescence images of RCon and ΔRI sporozoites on glass slides fixed with paraformaldehyde and stained with α-NH₂ or α-COOH terminal antisera. Shown in the top right is the percentage of 200 sporozoites staining with each respective antiserum. Bar=10 μm. FIG. 18B shows cleavage of the NH₂-terminus leads to exposure of the COOH-terminus. Wild type, RCon or ΔRI sporozoites were added to Hepa 1-6 cells for the indicated times, fixed and stained with α-NH₂ or α-COOH terminal antiserum or mAb 3D11. Shown is the percentage of total sporozoites staining with each antiserum. Shown are the means of triplicate coverslips with standard deviations. For each coverslip over 100 sporozoites were counted;

FIGS. 19A and 19B show anti-repeat region antibodies inhibit CSP cleavage and sporozoite invasion. Wild type P. berghei (FIG. 19A) or P. falciparum (FIG. 19B) sporozoites were metabolically labeled with ³⁵S-Cys/Met and then placed on ice (Control 0) or chased for 2 hr in the absence (Control 2) or presence of monoclonal antibody (3D11 or 2A10) at the indicated concentration (μg/ml) or in the presence of α-NH₂ terminal antiserum (α-N) at the indicated dilution. Sporozoites were then lysed, CSP immunoprecipitated and analyzed by SDS-PAGE and autoradiography. Invasion assays were performed with wild type P. berghei (FIG. 19A) or P. falciparum (FIG. 19B) sporozoites in the absence or presence of antibody at the indicated concentration or dilution and the percentage of intracellular sporozoites was determined. 50 fields were counted and shown are the means of triplicate wells±standard deviations. The experiment was repeated twice with identical results;

FIGS. 20A-20C show experiments related to antibodies to the minor repeats; and

FIG. 21 shows how CSP was generated in which region I was deleted.

### DESCRIPTION OF THE INVENTION

Generally, the present invention provides a composition and related methods for preventing or treating malaria infection. Specifically, the present invention is based on affecting and/or targeting the sporozoite stage of the parasite. More specifically, the present invention provides inhibitors that can inhibit sporozoite infection and thereby completely prevent malaria infection.

As used herein, the phrase, “malaria infection” means an infectious febrile disease caused by protozoa of the genus Plasmodium, which is transmitted by the bites of infected mosquitoes of the genus Anopheles. Malaria infection can be caused by any Plasmodium protozoa, including, but not limited to, Plasmodium vivax and Plasmodium falciparum.

The term “effective amount” as used herein, means, but is not limited to, the amount determined by such consideration as are known in the art of preventing or affecting malaria infection. The effective amount must be sufficient to provide an effect on malaria infection such as the elimination of infection or reduction thereof, which results in the elimination, reduction, or prevention of malaria symptoms or other measurements as appropriate and known to those of skill in the medical arts.

The term “protease inhibitor” as used herein includes, but is not limited to, peptide epoxides, of which L-trans-epoxysuccinyl-leucylamide-[4-guanido]-butane (E-64) is the prototype, Phenylmethylsulphonylfluoride (PMSF), Leupeptin, fluoromethyl ketones, acyloxymethyl ketones, chloromethyl ketones, peptide diazomethanes, allicin, combinations thereof, and any other similar protease inhibitor known to those of skill in the art. The present invention can utilize cysteine protease inhibitors. The protease inhibitor prevents proteolytic cleavage of sporozoites' CSP by a papain-family cysteine protease. The cleavage occurs on the sporozoite's cell surface. Cleavage of the CSP necessarily occurs during target/host cell invasion and is temporally associated with cell contact. Therefore, inhibitors of CSP processing inhibit cell invasion of the sporozoite in vitro and in vivo. Cysteine protease inhibitors are further described in the Examples below.

The terms “steric inhibitor” and “direct inhibitor” as used herein include, but are not limited to, a composition which, because of its size and/or shape, can alter one molecule's ability to chemically react with another molecule or with other parts of the same molecule. Steric and direct inhibitors interact with a molecule through various means, such as, but not limited to, chemical reactions, covalent bonds, cysteine bonds, hydrogen bonds, or other molecular forces. A steric inhibitor as used herein interacts with a region close to a site of reactivity on a molecule in order to alter the ability of the molecule to chemically react. A direct inhibitor as used herein refers specifically an inhibitor that interacts directly at a site of reactivity on a molecule in order to alter the ability of the molecule to chemically react.

The term “subject” as used herein includes, but is not limited to, humans, and any other similar subject capable of contracting and developing a malaria infection.

The present invention is based on the discovery that the high molecular weight circumsporozoite protein (hereinafter, “CSP”) form is proteolytically cleaved by a protease, specifically a papain-family cysteine protease, which gives rise to the low molecular weight form. The protease that cleaves CSP is of parasite origin and cleavage occurs on the sporozoite's surface. Cleavage necessarily occurs during target cell invasion and is temporally associated with cell contact. Inhibitors of CSP processing inhibit cell invasion in vitro and in vivo.

The present invention has numerous embodiments. In one embodiment, the present invention provides a composition for the prophylaxis of malaria infection. Preferably, the composition is a steric or direct inhibitor that targets the minor repeat region or region I of CSP. By binding to either the cleavage site of region 1 or to the minor repeats that are adjacent to the cleavage site, the inhibitor effectively blocks cleavage of CSP due to steric or direct hindrance of the protease binding to its cleavage site. The steric inhibitor refers to a compound which targets the minor repeat region, whereas the direct inhibitor refers specifically to a compound which targets region I.

The steric or direct inhibitor can be any suitable composition that is able to sterically hinder or block either the minor repeat region or region I of CSP from cleavage by a protease. The inhibition can be accomplished by the size and/or shape of the inhibitor blocking the cleavage region. As further described herein, the steric or direct inhibitor is preferably an antibody; however, other compounds and molecules with the required property of creating hindrance when binding or associating with CSP can also be used. Further, several antibodies are described herein, such as mAb 3D11, mAb 2A10, and mAb 2E6; however, any other suitable antibody can also be used.

One advantage of the present invention is that the antibodies used as steric or direct inhibitors are administered in low titers instead of high titers as has previously been done and deemed to be ineffective. Furthermore, targeting the minor repeats is more effective than targeting the major repeats of CSP, and thus lower quantities of antibodies or a steric or direct inhibitor in general are required to be effective in preventing malaria. These findings are further described in more detail below.

The present invention also provides for a pharmaceutical composition for preventing malaria infection including an effective amount of the inhibitor as described above and a pharmaceutical carrier. Preferably, the steric inhibitor targets a minor repeat region of CSP, and even more preferably, the direct inhibitor targets the cleavage site, namely region I. For the pharmaceutical composition to be effective, the inhibitor must prevent cleavage of CSP. Dosing and administration of the inhibitor is further described below.

The present invention provides for a method of malaria infection prophylaxis including the step of administering an effective amount of the composition, i.e. a steric or direct inhibitor as described above. The prophylaxis occurs through targeting a minor repeat region of CSP with the steric inhibitor. Or, preferably, region I is targeted by a direct inhibitor. Administration can be performed by medical personnel in a hospital setting or in an outpatient setting. Further, administration can preferably occur before exposure to malaria, such as before traveling to a malaria-infested region.

The following methods are accomplished by use of the inhibitors of the present invention in order to sterically or directly inhibit cleavage of CSP from occurring by targeting the minor repeat region, or specifically region I, and thus preventing malarial infection. For example, a method of malaria prophylaxis is provided including the step of inhibiting circumsporozoite protein processing by inhibiting cleavage of a circumsporozoite protein by an inhibitor. A method of malaria prophylaxis is provided including the step of inhibiting an interaction between a protease of a sporozoite and circumsporozoite protein. A method of preventing sporozoite cell invasion is provided including the step of administering an effective amount of the composition of the present invention. A method of preventing circumsporozoite processing is provided including the step of administering an effective amount of the composition of the present invention. A method of preventing malaria infection is provided including the step of preventing sporozoite cell invasion of a host cell by inhibiting circumsporozoite protein processing. The inhibiting step includes inhibiting cleavage of the sporozoite's circumsporozoite protein by a steric or direct inhibitor. More detail regarding these methods can be found below and in the Examples.

The conservation of region I in CS proteins from all species of Plasmodium suggests that it performs an important function in the life of the sporozoite. In this study, Applicants have identified that function and demonstrate that region I contains a proteolytic cleavage site required for hepatocyte invasion. The 5 amino acid sequence that defines region I is KLKQP.

CSP is a multifunctional protein with several critical and distinct functions during the sporozoite's life (reviewed in (Menard, 2000; Sinnis and Coppi, 2007). A role for CSP in sporozoite development and following this, in sporozoite egress from oocysts was demonstrated with mutants in which the CSP gene was deleted or altered. Deletion of CSP results in parasites that do not produce sporozoites (Menard et al., 1997) whereas altering the basic residues of the TSR results in the generation of sporozoites that cannot exit the oocyst (Wang et al., 2005). Although the precise role of CSP in sporozoite development and egress is not yet known, our data demonstrate that NH₂-terminal cleavage of the protein is not required for either process.

After sporozoites exit from oocysts, they must adhere to and enter salivary glands and Applicants' previous studies suggest a role for CSP in this process (Mo Myung et al., 2004; Sidjanski et al., 1997). CSP binds specifically to mosquito salivary glands and a peptide from the NH₂-terminal portion of CSP can inhibit this binding as well as sporozoite invasion of glands. The identity of the inhibitory peptide, which included region I as well as a stretch of upstream basic residues, raised the possibility that region I was involved in this process (Sidjanski et al., 1997). However, the lack of activity of short region I peptides (Sidjanski et al., 1997) as well as some longer peptides that included region I (Mo Myung et al., 2004) led Applicants to conclude that in the native protein clusters of basic residues from noncontiguous portions of the NH₂-terminus come together and mediate binding to salivary glands (Mo Myung et al., 2004). The lack of a significant role for region I in salivary gland invasion is supported by the current data demonstrating that sporozoites in which CSP has been engineered so that region I is deleted (hereinafter referred to “ΔRI sporozoites”) invade salivary glands at levels close to that observed with wild type parasites. The small but reproducible decrease in numbers of ΔRI salivary gland sporozoites could mean that the basic residues found in region I contribute to salivary gland binding or that proteolytic processing of CSP may have a minor role in this event.

In contrast to its minimal role in salivary gland invasion, CSP cleavage plays a critical role in sporozoite invasion of hepatocytes. The data herein demonstrates that the function of NH₂-terminal cleavage is to expose the COOH-terminal cell-adhesive TSR. It has been previously shown that both the TSR and the NH₂-terminal portion of CSP bind to HSPGs (Frevert et al., 1993; Rathore et al., 2002; Sinnis et al., 1994) and the findings herein suggest a model that explains why the CSP has two heparin-binding sequences. Applicants hypothesize that an initial low affinity interaction between HSPGs and the exposed NH₂-terminal portion of CSP cross links the protein and provides the signal for secretion of the protease. Removal of the NH₂-terminus of CSP then exposes the TSR which binds with high affinity to HSPGs, leading to productive invasion of the hepatocyte. Recently Applicants have shown that only cells expressing highly sulfated HSPGs are able to signal to the sporozoite to cleave CSP and productively invade and that this event is mediated in part by a sporozoite calcium-dependent protein kinase (CDPK-6; (Coppi et al., 2007)). Since in the mammalian host only hepatocytes express highly sulfated HSPGs, our data suggest a mechanism by which sporozoites retain their infectivity for an organ that is far from their site of entry.

ΔRI sporozoites, however, are not completely inhibited in their ability to invade hepatocytes. Although it is possible that a subset of sporozoites can invade hepatocytes using a CSP-cleavage-independent pathway, the more likely explanation for the residual infectivity of ΔRI sporozoites is the small amount of CSP cleavage observed in these mutants, enabling them to invade hepatocytes with a low efficiency. The data showing that metabolically-labeled ΔRI sporozoites do not cleave CSP in the absence of cells (during the 4 hour chase) but do cleave CSP to a limited degree when they contact hepatocytes, supports this hypothesis. It is likely that the protease, while preferring to cleave within region I, can cleave elsewhere in the N-terminus and when region I is absent, one of the several stretches of basic residues found in the NH₂-terminus of CSP could serve as an alternative cleavage site.

In contrast to Applicants' findings, a previous study in which region I of CSP was deleted found no effect on sporozoite infectivity in vivo (Tewari et al., 2002). In that study, however, the deletion was introduced into a P. berghei line in which the endogenous CSP gene was replaced by the P. falciparum CSP (PfCS). PfCS parasites, however, had 10-fold lower infectivity for mosquito salivary glands compared to the wild type P. berghei clone from which they were generated. These data suggest that the foreign P. falciparum CSP is not folded correctly in the rodent malaria parasite and can explain the difference between the studies.

Development of a malaria vaccine is a priority given the increasing incidence of malaria in the world (Greenwood, 2005). Immunity to the preerythrocytic stages of Plasmodium can be generated with high doses of irradiated sporozoites (Hoffman et al., 2002) and subsequent work has demonstrated that CSP is an important component of this protective response (Kumar et al., 2006). High titers of antibodies to the repeat region of CSP can inhibit sporozoite infectivity (Hollingdale et al., 1984; Hollingdale et al., 1982; Potocnjak et al., 1980) and may explain a portion of the protection afforded by irradiated sporozoites. However, these high titers have been difficult to achieve using peptides or proteins representing the repeats and an effective vaccine has not yet been achieved. Herein it is shown that antibodies directed against the repeat region of CSP inhibit proteolytic processing of CSP, a process that is required for hepatocyte invasion. The correlation between the antibody titers required to inhibit CSP cleavage and those required to inhibit hepatocyte invasion, suggests that inhibition of CSP cleavage is the mechanism by which anti-repeat antibodies inhibit sporozoite infectivity and suggest that inhibition of CSP cleavage may represent an in vitro assay for protective antibodies. Applicants have found that antibodies that target the minor repeats, which are closer to the cleavage site, are ten times more effective in inhibiting both CSP cleavage and sporozoite invasion of hepatocytes than antibodies targeting the major repeats which are further away from the cleavage site. Therefore, antibodies that directly target this cleavage site would be predicted to be most effective in inhibiting sporozoite infectivity than ever previously thought possible.

Given the proximity of the CSP repeats to region I, it is likely that anti-repeat antibodies sterically inhibit the interaction between the protease and CSP. The size of the central repeat region explains why large amounts of anti-repeat antibodies are required to inhibit sporozoite infectivity since the majority of these antibodies would bind to regions of the repeats distal to the cleavage site. The ineffectiveness of antiserum generated to a linear peptide representing the NH₂-terminal portion of CSP suggests that the cleavage site itself has a conformation that is not recognized by this antiserum and explains its inactivity in both sporozoite infectivity and CSP cleavage assays. The data herein raises the possibility that generation of antibodies specifically targeting the cleavage site would be effective at low titers and may present a new approach to preerythrocytic vaccine development. Further, because the cleavage site of CSP is highly conserved, the antibodies of the present invention are predicted to inhibit sporozoites from all species of malaria parasites. This is in contrast to vaccines that target the repeats which are species specific and so would only protect against a single Plasmodium species.

In conclusion, this is the first instance of a cleavage site mutant in an Apicomplexan protein, enabling the determination the function of CSP cleavage at each step of the sporozoite's journey. The data herein shows that NH₂-terminal processing of CSP is required for sporozoite entry into hepatocytes. The role of CSP cleavage in hepatocyte infection opens up new possibilities for inhibiting sporozoite infectivity in the mammalian host and thereby preventing malaria infection. These findings are further detailed in the Examples below.

Either the composition or the pharmaceutical composition of the present invention is administered and dosed in accordance with good medical practice, taking into account the clinical condition of the individual subject, the site and method of administration, scheduling of administration, subject age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement, prevention, or elimination of symptoms and other indicators as are selected as appropriate measures by those skilled in the medical arts.

The composition or the pharmaceutical composition of the present invention can be administered in various ways. It should be noted that it can be administered as the compound or as a pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Generally, the subject being treated is a warm-blooded animal and, in particular, mammals including man. The pharmaceutically acceptable carriers, diluents, adjuvants and vehicles as well as implant carriers generally refer to inert, non-toxic solid or liquid fillers, diluents, or encapsulating material not reacting with the active ingredients of the invention.

It is noted that humans are treated generally longer than mice or other experimental animals exemplified herein, which treatment has a length proportional to the length of the disease process, subject species being treated, and compound or drug effectiveness. The doses can be single doses or multiple doses over a period of several days, but single doses are preferred.

When administering the composition of the present invention parenterally, it can generally be formulated in a unit dosage injectable form (solution, suspension, or emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives that enhance the stability, sterility, and isotonicity of the compositions, including antimicrobial preservatives, antioxidants, chelating agents, and buffers, can be added. Prevention of the action of microorganisms can be ensured by various antibacterial and antifungal agents. Examples of these agents include, but are not limited to, parabens, chlorobutanol, phenol, sorbic acid, and the like. In many cases, it can be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicles, adjuvants, additives, and diluents. The compounds utilized in the present invention also can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques, which deliver it orally or intravenously and retain the biological activity, are preferred.

In one embodiment, the compound of the present invention can be administered initially by intravenous injection to bring blood levels to a suitable level. The patients levels are then maintained by an oral dosage form, although other forms of administration, dependent upon the patient's condition and as indicated above, can be used. The quantity to be administered can vary for the patient being treated and can vary from about 100 ng/kg of body weight to 100 mg/kg of body weight per day and preferably can be from 1 mg/kg to 10 mg/kg per day.

The invention is further described in detail by reference to the following experimental examples. These examples are provided for the purpose of illustration only, and are not intended to be limiting unless otherwise specified. Thus, the present invention should in no way be construed as being limited to the following examples, but rather be construed to encompass any and all variations which become evident as a result of the teaching provided herein.

### EXAMPLES

### Materials and Methods

Chemicals and Reagents:

All chemicals were obtained from Sigma-Aldrich (St. Louis, Mo.) except for aprotinin, antipain, AEBSF, leupeptin, pepstatin, 3,4-dichloroisocoumarin (3,4-DCI), chymostatin and L-trans-epoxysuccinyl-leucylamide-[4-guanido]-butane (E-64), which were obtained from Roche Applied Science (Indianapolis, Ind.). Western blot reagents were purchased from Amersham Pharmacia Biotech (Piscataway, N.J.) and other secondary antibodies were from Sigma-Aldrich.

Parasites:

Plasmodium berghei and Plasmodium yoelii sporozoites were grown in Anopheles stephensi mosquitoes and were obtained from infected salivary glands on the day of the experiment.

Antibodies:

mAb 3D11, directed against the repeat region of P. berghei CSP (Yoshida et al., 1980), was conjugated to sepharose and biotinylated using D-biotinoyl-ε-aminocaproic acid-N-hydroxysuccinimide ester as outlined in the manufacturer's protocol (Roche Applied Science).

Allicin Preparation:

Allicin was prepared by passing the synthetic substrate alliin [(+)S-2-propenyl L-cysteine S-oxide] through an immobilized alliinase column. The concentration of allicin was confirmed by HPLC and it was stored in a dark tightly closed tube at 4° C. for less than 3 months.

Antibodies and Peptides:

mAb 3D11 is directed against the repeat region of P. berghei CSP (Yoshida, et al., (1980)); mAbs 2F6 (P. Sinnis, F. Zavala, M. Tsuji, unpublished data) and NYS1 (Charoenvit, et al. (1987)) are directed against the repeat region of P. yoelii CSP; and mAb 2A10 is directed against the repeat region of P. falciparum CSP (Nardin, et al. (1982)). For immunoprecipitations, mAbs 3D11 and 2A10 were conjugated to sepharose using the protocol outlined in (Harlow, et al. (1988)). For gliding motility assays, mAbs 3D11 and NYS1 were biotinylated using D-Biotinoyl-e-aminocaproic acid-N-hydroxysuccinimide ester (Roche) as outlined in (Harlow, et al. (1988)). Antisera to the N- and C-terminal thirds of P. berghei CSP were generated using peptides from Institute of Biochemistry, at the University of Lausanne. The sequence of the N- and C-terminal peptides are

(SEQ ID NO. 1)

GYGQNKSIQAQRNLNELCYNEGNDNKLYHVLNSKNGKIYIRNTVNRLLAD
APEGKKNEKKNKIERNNKLK
and

(SEQ ID NO. 2)

NDDSYIPSAEKILEFVKQIRDSITEEWSQCNVTVSGSIRVRKRKGSNKKA
EDLTLEDIDTEICKMDKCS,
respectively.

Rabbits were injected three times, at one month intervals. The first injection (200 my of peptide in complete Freund's adjuvant) was intramuscular and subsequent injections (50 mg peptide in incomplete Freund's adjuvant) were subcutaneous. Overlapping peptides and the repeat peptides were synthesized by Midwest Bio-Tech (Indianapolis, Ind.) and purified by reverse-phase HPLC. The sequence was confirmed by mass spectrometry.

Sporozoites.

P. berghei and P. yoelii sporozoites were grown in Anopheles stephensi mosquitoes. P. falciparum infected mosquitoes were obtained from the NMRC Malaria Program (United States Navy).

Sporozoites were dissected from mosquito glands on the day of the experiment and where indicated, were purified by passage through two 3 mm pore polycarbonate membranes (Whatman, Clifton, N.J.).

ELISAs:

Peptides were coated onto wells of Immunlon 2HB microtiter plates (model 3455, ThermoLabsystems, Frankline, Mass.) overnight at 4° C., blocked and antisera was added at the indicated dilutions. Binding of antisera was revealed with either anti-mouse or anti-rabbit immunoglobulin (Ig) conjugated to alkaline phosphatase followed by the fluorescent substrate, 4-methylumbelliferyl phosphate. Fluorescence was read in a Fluoroskan II plate reader.

Metabolic Labeling Studies:

P. berghei sporozoites were incubated in Dulbecco's Modified Eagle Medium (DMEM; Invitrogen, Carlsbad, Calif.) without cysteine (Cys) and methionine (Met), containing 1% BSA and 400 mCi/ml L-[35 S]-Cys/Met (Pro-Mix, Amersham Pharmacia) in a total volume of 200 μl for one hour at 28° C. They were then washed and resuspended in DMEM/BSA containing unlabeled Cys/Met at 28° C. for the indicated time in the presence or absence of the indicated protease inhibitor. Sporozoites were then lysed and CSP was immunoprecipitated and analyzed by autoradiography as outlined below.

When sporozoites were incubated with specific inhibitors, their concentrations were as follows: 10 mM E-64; 1 mM phenylmethylsulfonyl fluoride (PMSF); 0.3 mM aprotinin; 100 mM 3,4 DCI; 75 mM leupeptin; 100 mM TLCK; 1 mM pepstatin; 1 mM 1,10 phenanthroline; 5 mM EDTA; 0.5% sodium azide. To check for toxicity by propidium iodide (PI) staining, P. berghei sporozoites were incubated in the presence or absence of the various protease inhibitors for two hours at 28° C., PI was then added to a final concentration of 1 mg/ml for five minutes at 28° C. The sporozoites were washed and viewed under a fluorescent microscope.

For the pronase experiment, sporozoites were labeled in medium without BSA for 45 minutes at 28° C., washed, and resuspended in DMEM with unlabeled Cys/Met and cycloheximide (100 mg/ml) for 10 minutes at 28° C. Then, they were kept on ice or chased at 28° C. for one hour Sporozoites were then resuspended in pronase (100 mg/ml) with or without pronase inhibitor cocktail [500 mg/ml antipain, 30 mg/ml aprotinin, 600 mg/ml chymostatin, 5 mg/ml EDTA, 5 mg/ml leupeptin, 10 mg/ml AEBSF, 7 mg/ml pepstatin, 2 mM PMSF; (Wieckowski, et al. (1998))] for one hour at 4° C., washed with pronase inhibitor cocktail, lysed in buffer supplemented with pronase inhibitor cocktail and 1% BSA, and CSP was immunoprecipitated and analyzed as outlined below.

Immunoprecipitation and SDS-PAGE Analysis:

Metabolically-labeled sporozoites were lysed in lysis buffer (1% Triton X-100, 50 mM Tris-HCl pH 8.0) with 150 mM NaCl containing a protease inhibitor cocktail (Complete Mini-Tablets, Roche) for one hour at 4° C. The lysates were incubated with mAb 3D11 conjugated to agarose overnight at 4° C. with agitation, and the beads were then washed sequentially with lysis buffer containing 150 mM NaCl, high salt buffer (500 mM NaCl in lysis buffer), lysis buffer without added NaCl, and pre-elution buffer (0.5% Triton X-100, 10 mM Tris-HCl pH 6.8). CSP was eluted with 1% SDS in 0.1 M glycine pH 1.8, neutralized with 1.5 M Tris-HCl pH 8.8, and run on a 7.5% SDS-polyacrylamide gel under nonreducing conditions.

For experiments with P. falciparum, a 10% SDS-polyacrylamide gel was used. Gels were fixed in 25% methanol/12% acetic acid, enhanced with Amplify (Amersham Pharmacia) for 30 minutes, dried, and exposed to film.

Immunoblot of Sporozoite Lysates:

Lysates of 5×10⁴ P. berghei sporozoite equivalents were loaded onto each lane of a 7.5% SDS-polyacrylamide gel under non-reducing conditions. Proteins were transferred to PVDF membrane and incubated with either mAb 3D11 (4 mg/ml), N-terminal antiserum (1:3000) or C-terminal antiserum (1:3000), followed by anti-mouse or anti-rabbit Ig conjugated to horseradish peroxidase (HRP; 1:100,000). Bound antibodies were visualized using the enhanced chemiluminescence detection system (ECL).

Biotinylation of Sporozoites:

P. berghei sporozoites expressing green fluorescent protein [GFP; (Natarajan, et al. (2001))] were biotinylated using sulfo-succinimidyl-6′-(biotinamido) hexanoate according to the manufacturer's instructions (Pierce, Rockford, Ill.). Lysates of biotinylated sporozoites were immunoprecipitated with either mAb 3D11 or polyclonal antibodies to GFP (1:200; Molecular Probes, Eugene, Oreg.) followed by Protein A coupled to agarose beads. Beads were washed and bound proteins were eluted according to the protocol outlined above. 5×10⁴ sporozoite equivalents were loaded onto each lane of a 4-12% Tris-Glycine gel (invitrogen) under nonreducing conditions, transferred to PVDF, and incubated with either mAb 3D11 followed by anti-mouse Ig conjugated to HRP, anti-GFP Ig (1:500), followed by anti-rabbit Ig conjugated to HRP, or streptavidin conjugated to HRP (1:100,000). Bound antibodies were visualized using ECL.

Immunofluorescence Assay:

Live P. berghei sporozoites were incubated with N-terminal antiserum (1:500 in DMEM/BSA) at 4° C. for two hours, washed three times at 4° C., and allowed to air dry on slides at 4° C. They were then incubated with anti-rabbit Ig-FITC, washed, and mounted in Citifluor (Ted Pella Inc., Redding, Calif.).

Sporozoite Invasion Assay:

Invasion assays were performed as previously described (Pinzon-Ortiz, et al. (2001) and Renia, et al. (1988)), with some modifications. For assays with P. berghei and P. yoelii, Hepa 1-6 cells (ATCC CRL-1830, American Type Culture Collection, Manassas, Va.), a mouse hepatoma cell line permissive for P. yoelii sporozoite development (Mota, et al. (2000)) was seeded (8×10⁴ cells/well) in Lab-Tek permanox chamber slides (Nalgene Nunc Corp., Naperville, Ill.) and grown until confluent.

For assays with P. falciparum, HepG2 cells (ATCC HB8065, American Type Culture Collection) were used. On the day of the experiment, sporozoites were pre-incubated with DMEM/BSA alone or with the indicated protease inhibitor for two hours at 28° C. and plated on cells in the continued presence of the inhibitor for one hour at 37° C.

In a control, Hepa 1-6 cells were incubated with E-64 for two hours at 37° C., the medium was removed and then untreated P. berghei sporozoites were added. The inhibitors used were: 10 mM E-64; 1 mM PMSF; 75 mM leupeptin; 0.3 mM aprotinin; 100 mM 3,4 DCI; 1 mM pepstatin. After incubation with sporozoites, cells were washed, fixed with 4% paraformaldehyde and sporozoites were stained with mAb 3D11 (P. berghei), mAb 2F6 (P. yoelii) or mAb 2A10 (P. falciparum) followed by anti-mouse Ig conjugated to rhodamine. Cells were then permeabilized with cold methanol and stained again with mAbs 3D11, 2F6 or 2A10 followed by anti-mouse Ig conjugated to FITC. All sporozoites were FITC-positive whereas only extracellular sporozoites stained with rhodamine. The percentage of sporozoites that invaded the cells is calculated using the following equation:

$$\% invasion = \frac{total sporozoites - extracellular sporozoites \times 100}{total sporozoites .}$$

Cell Contact Assay:

Hepa 1-6 cells were seeded on glass coverslips at a density of 2×10⁵ cells per coverslip and grown until confluent. P. berghei sporozoites were incubated in DMEM±10 mM E-64 at 4° C. for two hours. Thirty minutes before sporozoites were added to coverslips, Cytochalasin D (CD) was added to all samples (final concentration, 1 mM). Sporozoites were centrifuged onto coverslips (1250×g) for five minutes at 4° C. Coverslips were then brought to 37° C. for two minutes, fixed with 4% paraformaldehyde and stained with either mAb 3D11 followed by anti-mouse Ig FITC or the N-terminal antiserum (1:100) followed by anti-rabbit Ig FITC. When P. berghei sporozoites transgenic for GFP were used, the cells were only stained with the N-terminal antiserum followed by anti-rabbit Ig FITC. As a control, sporozoites were spun onto coverslips without cells using the protocol outlined above.

Sporozoite Motility Assay:

Lab-Tek glass slides (model 177402, Nalgene Nunc Corp., Naperville, Ill.) were coated with 10 mg/ml of mAb 3D11 (P. berghei) or mAbs 2F6 and NYS1 (P. yoelii) and sporozoites pre-incubated with DMEM/BSA alone or supplemented with 10 mM E-64 or CD for two hours at 28° C. were added in the continued presence of the inhibitor for one hour at 37° C. in a humidified chamber with 5% CO₂. The slides were then fixed with 4% paraformaldehyde, incubated with 1:100 dilution of either biotinylated mAb 3D11 (P. berghei) or biotinylated mAb NYS1 (P. yoelii) followed by streptavidin-FITC.

Sporozoite Migration Assay:

Migration assays were performed as previously described (Mota, M., et al.). Sporozoites were pre-incubated±10 mM E-64 for two hours at 28° C. and added to monolayers of Hepa 1-6 cells in the continued presence of inhibitor with 1 mg/ml rhodamine-dextran, 10,000 MW (Molecular Probes). After one hour at 37° C., the cells were washed, fixed and the number of rhodamine-positive cells in each field was counted.

Staining of Sporozoites in Dextran-Positive Cells:

P. berghei sporozoites were added to Hepa 1-6 cells in the presence of 1 mg/ml rhodamine-dextran for 45 minutes at 37° C., washed, and fixed with 4% paraformaldehyde. Extracellular sporozoites were stained with mAb 3D11 followed by anti-mouse Ig conjugated to 10 nm gold (1:50, Amersham Pharmacia). Hepa 1-6 cells were then permeabilized with 0.1% saponin, which does not allow the escape of intracellular dextran, and intracellular sporozoites were stained with the N-terminal antiserum (1:100) followed by anti-rabbit Ig FITC (1:500, Molecular Probes). Coverslips were washed and developed using the Intense M Silver Enhancement kit (Amersham Pharmacia) for fifteen minutes at room temperature.

Assay for Sporozoite Infectivity In Vivo:

Swiss/Webster mice were given three intraperitoneal injections of DMEM with or without E-64 (50 mg/kg/injection) at 16 hours, 2.5 hours, and 1 hour prior to intravenous injection of 15,000 P. yoelii sporozoites. Forty hours later, livers were harvested and total RNA was isolated using Tri-Reagent (Molecular Research Center, Cincinnati, Ohio). Malaria infection was quantified using reverse-transcription (RT) followed by real time PCR as outlined in (Bruna-Romero, et al. (2001)). RTs were performed with 0.5 mg of total RNA and random hexamers (PE Applied Biosystems, Foster City, Calif.). Real time PCR was performed using primers that recognize P. yoelii-specific sequences within the 18S rRNA (Bruna-Romero, et al. (2001)) and the SYBR Green Core PCR kit (PE Applied Biosystems). Ten-fold dilutions of a plasmid construct containing the P. yoelii 18S gene were used to create a standard curve.

Metabolic Labeling, Immunoprecipitation and SDS-Page Analysis:

P. berghei sporozoites were metabolically labeled as previously described (Bruna-Romero, O., et al.). Briefly, sporozoites were labeled in Dulbecco's Modified Eagle Medium containing 1% BSA (DMEM/BSA) without Cys/Met and with 400 μCi/ml L-[³⁵S]Cys/Met for one hour at 28° C. and chased in DMEM/BSA with Cys/Met at 28° C. in the presence of 10 μM E-64 or the indicated concentrations of allicin. Labeled sporozoites were lysed in 1% Triton X-100/150 mM NaCl/50 mM Tris-HCl pH 8.0 with protease inhibitors, and lysates were incubated with 3D11-sepharose overnight at 4° C. CSP was eluted with 1% SDS in 0.1 M glycine pH 1.8, neutralized with Tris-HCl pH 8.8, and run on a 7.5% SDS-polyacrylamide gel under nonreducing conditions. The gel was fixed, enhanced with Amplify (Amersham Pharmacia), dried and exposed to film.

Allicin Toxicity Assay:

P. berghei sporozoites were incubated with the indicated concentrations of allicin for ten or sixty minutes at 28° C., washed with DMEM, and then incubated with 1 μg/ml propidium iodide for five minutes at 25° C. The number of fluorescent sporozoites in each sample was counted using a Nikon Eclipse E600 microscope. Control samples consisted of sporozoites that were incubated for sixty minutes at 28° C. in DMEM without allicin and sporozoites that were heat killed at 65° C. for ten minutes.

Gliding Motility Assay:

Glass 8-chambered Lab-tek wells (Nalgene) were coated with 10 μg/ml 3D11 in PBS overnight at 25° C. and then washed three times with PBS. 2×10⁴ P. berghei sporozoites were incubated with 50 μM allicin in DMEM without Cys/Met for ten minutes at 28° C., the medium removed and replaced with DMEM/3% BSA containing 50 μM or 4.2 μM allicin before sporozoites were added to the coated Lab-Tek wells. The sporozoites were incubated for one hour at 37° C., the medium was removed, and the wells were fixed with 4% paraformaldehyde, washed, blocked with PBS/1% BSA, and incubated with biotinylated 3D11 followed by Streptavidin-FITC (1:100 dilution; Amersham Pharmacia). All incubations were performed at 37° C. for one hour. Controls included untreated sporozoites and sporozoites added to wells in the presence of 1 μM cytochalasin D. For each group, gliding motility was assessed by determining the percentage of sporozoites associated with trails, and for those sporozoites with trails, counting the number of circles in each trail.

Sporozoite Invasion Assays:

Invasion assays were performed as previously described (Pinzon-Ortiz, C., et al.) with some modifications. P. berghei sporozoites were preincubated with the indicated concentrations of allicin for ten minutes at 28° C., diluted 12-fold with DMEM/BSA and added to Hepa 1-6 cells (CRL-1830: American Type Culture Collection) for one hour at 37° C. Cells were then washed, fixed, and sporozoites were stained with a double staining assay that distinguishes between intracellular and extracellular sporozoites (Renia, L., et al.).

Assay for Sporozoite Infectivity In Vivo:

Swiss/Webster mice were given either 5 or 8 mg/kg of allicin (in DMEM without Cys/Met) intravenously (i.v.) at 60 minutes, 30 minutes, or immediately before i.v. injection of 10⁴ P. yoelii sporozoites. Forty hours later, livers were harvested, total RNA was isolated, and malaria infection was quantified using reverse transcription (RT) followed by real time PCR using primers that recognize P. yoelii-specific sequences within the 18S rRNA as previously described (Bruna-Romero, O., et al.). Ten-fold dilutions of a plasmid construct containing the P. yoelii 18S rRNA gene were used to create a standard curve.

For allicin preincubation experiments, P. yoelii sporozoites were preincubated with or without 50 μM allicin (in DMEM without Cys/Met) for ten minutes at 28° C. and diluted 12-fold with medium before i.v. injection into mice. All in vivo data were analyzed using the Student t-test for unpaired samples.

The following materials and methods apply to Examples 9-18:

Parasites:

In Examples 9-18, 3 to 5 day-old Anopheles stephensi mosquitoes were fed on Swiss Webster mice infected with P. berghei ANKA strain wildtype, RCon or ΔRI erythrocytic-stage parasites. On days 10 through 22 post-infective blood meal, midguts, salivary glands and hemolymph were harvested for determination of sporozoite numbers. For midgut and salivary gland counts, mosquitoes were anesthetized on ice, rinsed in 70% ethanol, washed in Dulbecco's Modified Eagle Medium (DMEM), and 20 of each organ were pooled, homogenized to release sporozoites and centrifuged (80×g) to remove mosquito debris. Hemolymph sporozoites were collected by perfusion of the mosquito thorax and abdomen with 20 μls of DMEM. Hemolymph from 20 mosquitoes was pooled and counted. For trypsin experiments salivary glands were incubated in DMEM with 50 μg/ml trypsin (Sigma) for 15 min at 37° C., centrifuged for 5 min at 80×g to pellet the glands and then sporozoites in supernatant (containing attached sporozoites) and salivary glands (containing invaded sporozoites) were counted. In all cases, sporozoites were counted in a hemocytometer. Purified and irradiated P. falciparum sporozoites were generously provided by Dr. Stephen L. Hoffman (Sanaria Inc., Rockville, Md.).

Generation of ΔRI and RCon Parasites:

Recombinant P. berghei parasites (ANKA strain) were generated by double homologous recombination using the targeting vector pCSRep (see Supplemental Materials and Methods for details of plasmid construction) in which a wild type or mutant copy of CSP, with its upstream and downstream control elements, was placed downstream of the selection cassette which includes the human dihydrofolate reductase/thymidilate synthase gene (hDHFR) flanked by the upstream and downstream control elements from P. berghei DHFR/TS. This cassette confers resistance to pyrimethamine and was targeted to the CSP locus with additional CSP 5′UTR placed upstream of the selection cassette. The plasmid was digested with XhoI and EcoRI to release the fragment for transfection. P. berghei schizonts collected from Wistar rats were electroporated with 5 μg of DNA using the Amaxa Nucleofector (program U33) as previously outlined (Janse et al., 2006). Selection and cloning by limiting dilution were performed in mice as previously described (Menard and Janse, 1997). Integration of the transfected DNA at the correct location was verified for each RCon and ΔRI clone by both PCR and Southern blot. PCR to verify integration at the CSP locus was performed with primers P1 (5′-AATGAGACTATCCC TAAGGG-3′) and P2 (5′-TAATTATATGTTATTTTATTTCCAC-3′). Southern blotting was performed with genomic DNA from either RCon or ΔRI erythrocytic stage parasites digested with EcoRV and probed with the 873 bp PmlI-PacI fragment of CSP which was labeled with digoxigenin-dUTP by random priming and detected using the DIG High Prime DNA Labeling and Detection Kit (Roche). In addition, the CSP coding sequence of each clone was amplified using primers P3 (5′-CGAGCTATGTTACAATGAAGG-3′) and P4 (5′-AAATTCTAGTATTTTTTCCGCGC-3′) and sequenced to confirm that the deletion was not corrected in ΔRI parasites and that the repeats remained unchanged for both mutant and control recombinant parasites.

Cells and Antibodies:

Hepa 1-6 (CRL-1830; ATCC, Rockville, Md.) and HepG2 cells (HB-8065; ATCC) were maintained in DMEM supplemented with 10% fetal calf serum (FCS) and 1 mM glutamine (DMEM/FCS). mAb 3D11 is directed against the repeat region of P. berghei CSP (Yoshida et al., 1980); mAb 2A10 is directed against the repeat region of P. falciparum CSP (Nardin et al., 1982); mAb 2E6 is directed against the Plasmodium Hsp 70 (Tsuji et al., 1994). Polyclonal antisera to the NH₂— and COOH-terminal portions of P. berghei CSP were generated using peptides that were generously provided by Drs. Giampietro Corradin and Mario Roggero (Institute of Biochemistry, University of Lausanne) as outlined in (Coppi et al., 2005). Polyclonal antisera to the P. berghei TRAP repeat region was generated by immunization of rabbits with peptide conjugated to KLH (P. Sinnis, unpublished data).

Immunoblot of Sporozoite Lysates:

Sporozoites were lysed in nonreducing sample buffer and 5×10⁴ sporozoite equivalents/lane were loaded and separated by SDS-PAGE, transferred to PVDF membrane and incubated with either mAb 3D11 (4 μg/ml), or rabbit polyclonal antisera directed against the TRAP repeats (1:200) followed by anti-mouse or anti-rabbit Ig conjugated to horseradish peroxidase (HRP; 1:100,000). Bound antibodies were visualized using the enhanced chemiluminescence detection system.

Immunofluorescence:

RCon and ΔRI sporozoites from salivary glands were fixed with 4% paraformaldehyde, washed, blocked with PBS/BSA and incubated with mAb 3D11 (1 μg/ml) followed by anti-mouse Ig conjugated to FITC. Specimens were mounted in Citifluor (Ted Pella Inc., Redding, Calif.) and photographed using a Nikon E600 Fluorescence Microscope and a DXM1200 digital camera.

Metabolic Labeling and Immunoprecipitation:

P. berghei sporozoites were metabolically labeled in DMEM without Cys/Met, 1% BSA and 400 μCi/ml L-[³⁵S]-Cys/Met for 1 hour at 28° C. and then kept on ice or chased at 28° C. for the indicated times. Details of this procedure are outlined in (Coppi et al., 2005). To investigate the kinetics of processing in the presence of hepatocytes, sporozoites were labeled as above, chased at 28° C. for 1 hour and then centrifuged (300×g) onto glass coverslips with Hepa 1-6 cells and incubated at 37° C. for 5, 15 or 30 minutes. To investigate the effect of antibodies on CSP cleavage, sporozoites were labeled as above and then chased for 2 hours in the presence of the indicated antibody. Monoclonal antibodies were used as purified antibodies at the indicated concentrations and the IgG fraction of the NH₂-terminal antiserum was used after isolation by Protein A (Vivapure maxiprepA; Sartorius, Edgewood, N.Y.). In all cases, labeled sporozoites (with or without Hepa 1-6 cells) were lysed and labeled CSP was immunoprecipitated with mAb-3D11 conjugated to sepharose or in the case of P. falciparum CSP, with mAb-2A10 conjugated to sepharose, as previously described (Coppi et al., 2005). CSP was eluted from the beads, run on an SDS-PAGE gel which was enhanced, dried and exposed to film.

Cell Contact and CSP Cleavage Assay:

Sporozoites were centrifuged onto coverslips containing Hepa 1-6 cells at 4° C. and then incubated at 37° C. for the indicated time points, fixed with 4% paraformaldehyde and stained with either mAb 3D11 or antiserum recognizing the NH2-terminus or the COOH-terminus of CSP. The total number of sporozoites was determined using mAb 3D11 which stains all sporozoites and the fraction of those staining with each antiserum was determined.

Invasion and Development Assays:

Hepa 1-6 cells were seeded in Permanox eight-chambered Lab-Tek wells (2.5×10⁵/well) and allowed to grow overnight. On the day of the experiment, 5×10⁵ P. berghei wild type, RCon or ΔRI sporozoites were added per well. Sporozoites pre-incubated with 10 μM E-64d for 15 minutes at 25° C. were used as controls. For experiments testing the effect of anti-repeat region antibodies on invasion, HepG2 cells were used and P. berghei or P. falciparum sporozoites were added to the cells in the presence of the indicated antibody. After 1 hour at 37° C., cells were washed, fixed, and sporozoites were stained with a double staining assay that distinguishes intracellular from extracellular sporozoites (Renia et al., 1988). To quantify EEF development, cells with sporozoites were grown for an additional 2 days after which they were fixed with methanol, stained with mAb 2E6 followed by goat anti-mouse Ig conjugated to FITC. In all assays, at least 50 fields per well were counted and each point was performed in triplicate.

Gliding Motility Assay:

P. berghei wild type, RCon and ΔRI sporozoites were added to Lab-Tek wells coated with mAb 3D11 as previously outlined (Coppi et al., 2005). After 1 hour at 37° C., the medium was removed and the wells were fixed and stained with biotinylated mAb 3D11 followed by streptavidin-FITC (1:100 dilution; Amersham Pharmacia) in order to visualize the CSP-containing trails. Gliding motility was quantified by counting the number of sporozoites associated with trails and for those sporozoites with trails, counting the number of circles in each trail. Over 200 sporozoites per well were counted and each point was performed in triplicate.

Calcein Migration Assay:

Hepa 1-6 cells were plated in 96-well tissue culture-treated plates, allowed to grow until semi-confluent, washed with DMEM without phenol red, loaded with 10 μM calcein green AM for 1 hour at 37° C. and washed 3 times. 5×10⁵ sporozoites in culture medium were centrifuged onto the calcein-loaded cells, incubated for 1 hour at 37° C. and supernatants containing the released calcein were transferred to a ThermoElectron Microfluor 96-well plate. Fluorescence was read in a Labsystems Fluoroscan II using excitation and emission wavelengths of 485 nm and 538 nm, respectively. Further details are outlined in (Coppi et al., 2007).

Quantification of Liver Stage Burden:

4 to 5 week old female Swiss Webster and C57BI/6 mice were injected intravenously with 104 sporozoites. 40 hour later, livers were harvested, total RNA was isolated, and liver parasite burden was quantified by reverse transcription followed by real-time PCR as outlined previously (Bruna-Romero et al., 2001) with some modifications. PCR was performed using primers that recognize P. berghei-specific sequences within the 18S rRNA (Kumar et al., 2004) and the temperature profile of the real-time PCR was 95° C. for 15 minutes, followed by 40 cycles of 95° C. for 30 seconds, 58° C. for 30 seconds and 72° C. for 30 seconds. Ten-fold dilutions of a plasmid construct containing the P. berghei 18S rRNA gene were used to create a standard curve.

Determination of Prepatent Period:

Swiss Webster or CS7BI/6 mice were injected intravenously or intradermally with 5000 sporozoites and the onset of blood stage infection was determined by performing daily blood smears, beginning 3 days post-infection. Blood smears were stained with giemsa and scanned under high power for erythrocytic stage parasites.

### Example One

### The N-Terminal Portion of CSP is Proteolytically Cleaved by a Cysteine Protease

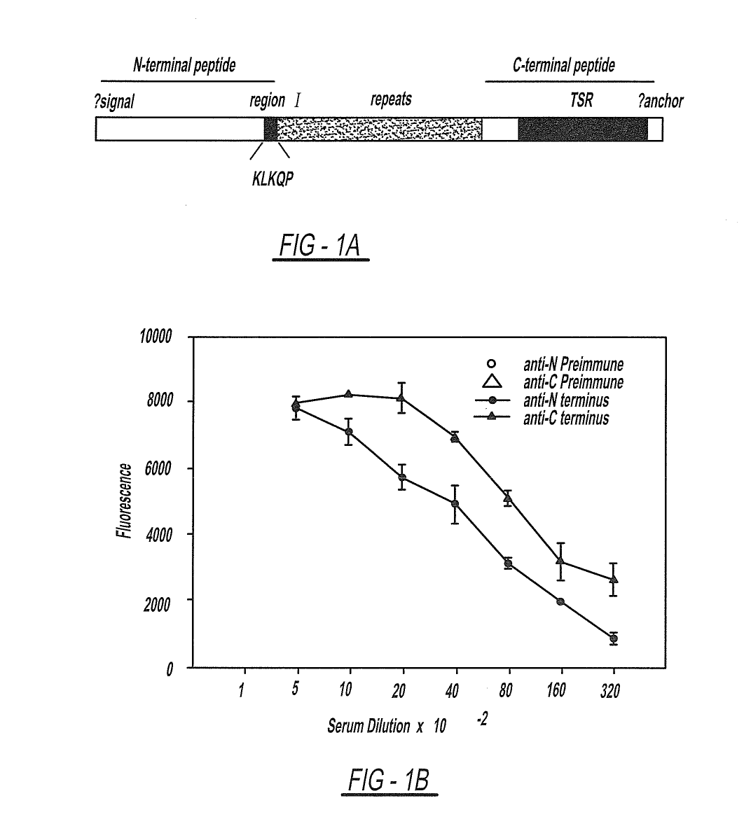

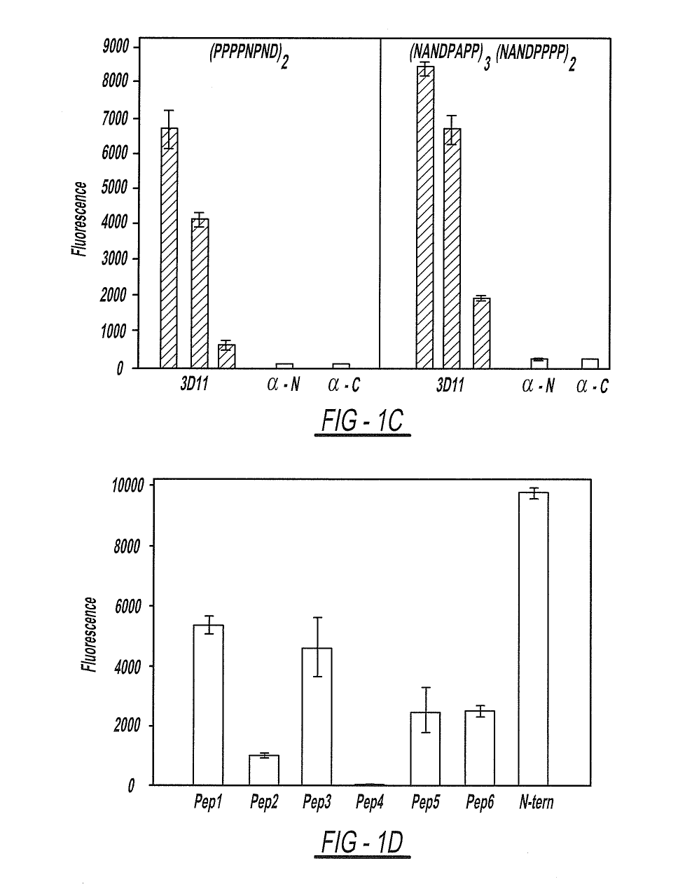

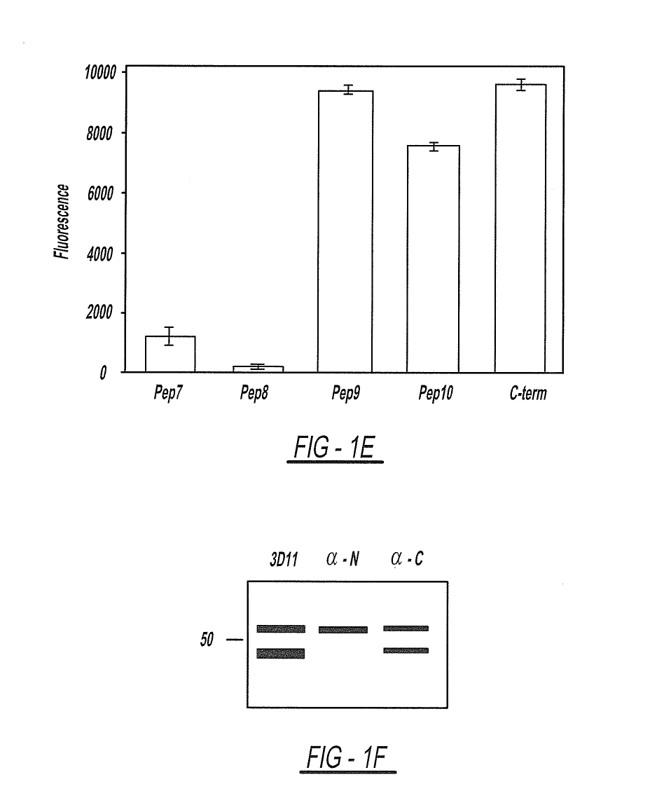

As shown in FIG. 1, Panel A represents that CSPs from all species of Plasmodium have the same overall structure. There is a central species-specific repeat region (grey box) and two conserved stretches of amino acids (black boxes); a 5 amino acid sequence called region I and a cell-adhesive sequence with similarity to the type I thrombospondin repeat (TSR; (Goundis, D., et al.)). The first 20 residues of CSP have the features of a eukaryotic signal sequence (Nielsen, H., et al.) and the C-terminal sequence can contain an attachment site for a lipid anchor (Moran, P., et al.). Bars show the location of peptides used for the generation of antisera. For Panels B-E, they illustrate that rabbits were immunized with the long N-terminal or C-terminal peptides and sera were tested for specificity by ELISA. All points were performed in triplicate and shown are the means with standard deviations. Specifically, Panel B illustrates dilutions of the N-terminal antiserum or C-terminal antiserum that were tested for reactivity to full length N- and C-terminal peptides respectively. A 1:100 dilution of each preimmune serum was also tested for reactivity to the appropriate full-length peptide. Panel C illustrates that mAb 3D11 (1, 0.1 and 0.01 mg/ml from left most bar respectively) and 1:100 dilutions of the N-terminal antiserum and C-terminal antiserum were tested for reactivity to the indicated repeat peptides. Panel D shows that a 1:100 dilution of the N-terminal antiserum was tested for reactivity to a series of overlapping peptides encompassing the N-terminal third of CSP. Sequences of the peptides are: Pep 1: GYGQNKSIQAQRNLNE (SEQ ID NO. 3); Pep 2: RNLNELCYNEGNDNKL (SEQ ID NO. 4); Pep 3: NDNKLYHVLNSKNGKI (SEQ ID NO. 5); Pep 4: KNGKIYIRNTVNRLLA (SEQ ID NO. 6); Pep 5: NRLLADAPEGKKNEKK (SEQ ID NO. 7); and Pep 6: KNEKKNKIERNNKLK (SEQ ID NO. 8); N-term: full-length N-terminal peptide. Panel E illustrates a 1:100 dilution of the C-terminal antiserum was tested for reactivity to overlapping peptides encompassing the entire C-terminal third of CSP. Sequences of the peptides are Pep 7: NDDSYIPSAEKILEFVKQI (SEQ ID NO. 9); Pep 8: FVKQIRDSITEEWSQCNVT (SEQ ID NO. 10); Pep 9: QCNVTCGSGIRVRKRKGSNKKAEDL (SEQ ID NO. 11); Pep 10: KKAEDLTLEDIDTEICKM (SEQ ID NO. 12); C-term: full-length C-terminal peptide. Finally, Panel F illustrates a western blot of P. berghei sporozoite lysates that was performed using the anti-repeat region antibody mAb 3D11, the N-terminal antiserum, or the C-terminal-antiserum.

The antisera recognized the appropriate full-length peptides and did not recognize peptides representing the central repeat domain (FIG. 1C). In addition, the N-terminal antiserum did not recognize the C-terminal peptide and the C-terminal antiserum did not recognize the N-terminal peptide. To identify the epitopes recognized by each antiserum, their reactivity was tested to small overlapping peptides encompassed by the long parent peptides. As shown, the N-terminal antiserum recognized peptides interspersed throughout the N-terminal third of the protein (FIG. 1D). In contrast, the C-terminal antiserum only recognized peptides from the C-terminus of the full-length C-terminal peptide (FIG. 1E).

Western blot analysis of a P. berghei sporozoite lysate using the N- and C-terminal specific antisera (FIG. 1F) was then performed. As a control, the monoclonal antibody (mAb) 3D11, which recognizes the repeat region of P. berghei CSP (Yoshida, et al. (1980)), was used. As expected, mAb 3D11 recognized both CSP forms. However, the N-terminal antiserum recognized only the high molecular weight CSP form. Since this antiserum reacts with epitopes throughout the N-terminal third of CSP (FIG. 1D), the low molecular weight CSP form lacks this entire region.

The present data provides evidence that the conserved region I, found at the end of the N-terminal portion of CSP, contains the cleavage site. In order to determine what class of protease is responsible for cleavage, pulse-chase metabolic labeling experiments were performed in the presence of protease inhibitors. Previous studies showed that after one hour of labeling, radioactivity is found in the high molecular weight CSP form and at 28° C., the half-life of this species is between sixty to ninety minutes (Yoshida, et al. (1981) and Cochrane, et al. (1982)).

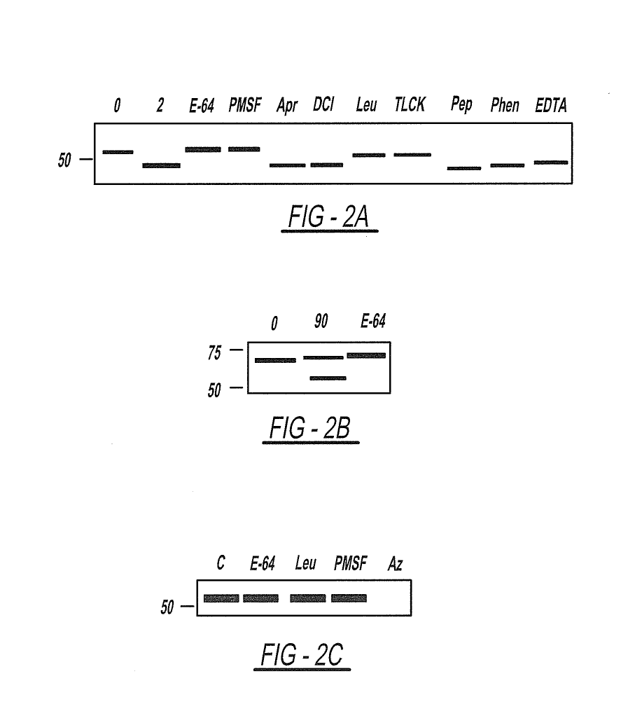

As supported by FIG. 2, CSP processing is inhibited by cysteine and some serine protease inhibitors. Panel A illustrates P. berghei sporozoites were metabolically labeled with [35 S]-Cys/Met and then washed and kept on ice (lane 1) or chased with cold medium for two hours at 28° C., in the absence (lane 2) or presence of the indicated protease inhibitors (lanes 3-11). After the chase, sporozoites were lysed, CSP was immunoprecipitated and analyzed by SDS-PAGE and autoradiography (abbreviations: Apr, aprotinin; DCI, 314 DCI; Leu, leupeptin; Pep, pepstatin; Phen, 1110 phenanthroline). Panel B illustrates P. falciparum sporozoites were metabolically labeled as above and then kept on ice (lane 1) or chased with cold medium for ninety minutes in the absence (lane 2) or presence of E-64 (lane 3). Samples were processed as outlined above. Panel C illustrates P. berghei sporozoites were preincubated with buffer (lane 1) or the indicated compounds, washed, metabolically labeled with [³⁵S]-Cys/Met, lysed, and CSP was immunoprecipitated and analyzed by SDS-PAGE and autoradiography. (Abbreviations: Az=sodium azide).

As set forth above, sporozoites were labeled with [35 S]-Cys/Met for one hour and chased with medium containing unlabeled Cys/Met for two hours in the presence or absence of protease inhibitors (FIG. 2A). In the absence of protease inhibitors, approximately 80% of labeled CSP is cleaved after two hours. In the presence of the metalloprotease inhibitor 1,10 phenanthroline or the aspartyl-protease inhibitor pepstatin, there was no effect on CSP processing. In addition, EDTA had no effect on CSP processing, indicating that divalent cations are not required. Leupeptin and TLCK, inhibitors of both cysteine and serine proteases, E-64, a highly specific cysteine protease inhibitor, and PMSF, a serine protease inhibitor, all inhibited CSP processing. Although PMSF has been reported to have inhibitory activity against some papain-family cysteine proteases (Whitaker, et al. (1968) and Solomon, et al. (1999)), it is a prototypical serine protease inhibitor.

To further examine the role of serine proteases, two other serine protease inhibitors, aprotinin and 3,4 DCI, were assayed. Aprotinin inhibits most classes of serine proteases and would be predicted to inhibit the serine proteases of Plasmodium, which are subtilisin-like serine proteases (Wu, et al. (2003)). 3,4 DCI is a serine protease inhibitor that has some activity against cysteine proteases, but does not react with papain-like cysteine proteases (Harper, et al. (1985)). Neither compound had an effect on CSP processing. Taken together, the data proves that the processing enzyme is a cysteine protease.

In addition to the above, pulse-chase metabolic labeling experiments were performed with sporozoites of the human malaria parasite, Plasmodium falciparum. As shown in FIG. 2B, E-64 inhibited CSP processing in this species. This data suggests that CSP cleavage occurs by a similar mechanism in both rodent and human Plasmodium species. In order to insure that the protease inhibitors were not toxic to sporozoites, sporozoites were incubated with the various inhibitors for two hours and then propidium iodide (hereinafter, “PI”) was added, which is a fluorescent molecule that enters permeabilized cells. The percentage of sporozoites that took up the dye in the presence of any of the protease inhibitors was no different from controls. Since uptake of PI is a terminal event, we also tested whether sporozoites incubated with protease inhibitors were less metabolically active. To do this, CSP synthesis, after sporozoites had been incubated with individual inhibitors for two hours, was analyzed. As shown, CSP synthesis was not affected by E-64, leupeptin or PMSF (FIG. 2C).

### Example Two

### CSP Cleavage Occurs Extracellularly by a Sporozoite Protease

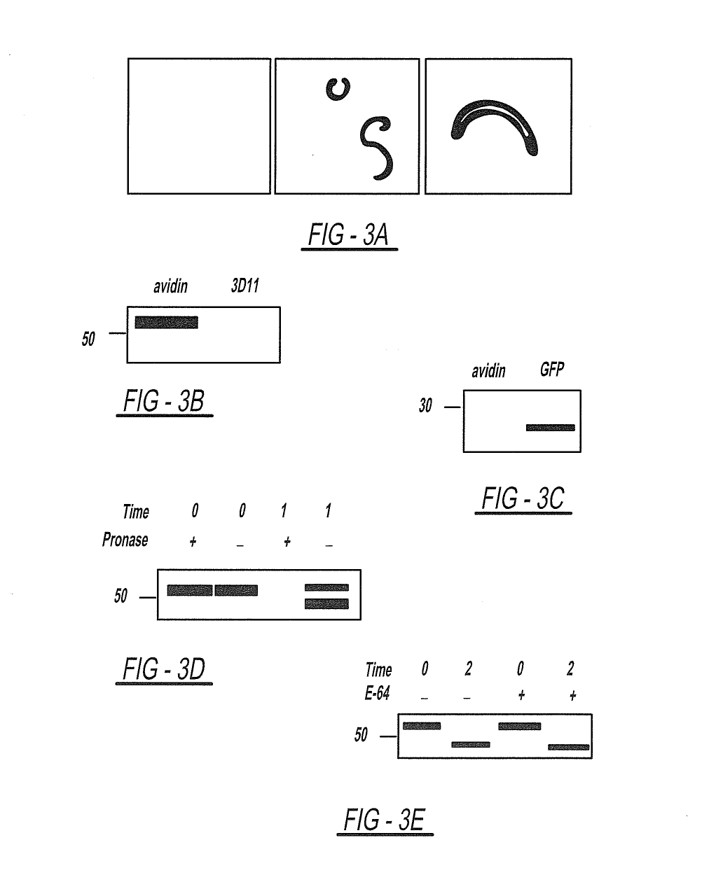

FIG. 3 illustrates that CSP is processed extracellularly by a parasite protease. As set forth in FIG. 3, Panel A shows that live sporozoites were incubated with the N-terminal antiserum followed by anti-rabbit Ig conjugated to FITC. Phase contrast (left) and fluorescence (center and right) views are shown (Bar=10 mm). Panels B & C show that P. berghei sporozoites expressing GFP were biotinylated, lysed, and CSP (panel B) and GFP (panel C) were immunoprecipitated from the lysate. A western blot of the immunoprecipitated material was probed with streptavidin (lane 1 of panels B & C), mAb 3D11 (lane 2, panel B) or polyclonal antisera to GFP (lane 2, panel C). Panel D shows P. berghei sporozoites were metabolically labeled, washed, and kept on ice (Time=0) or chased at 28° C. for one hour (Time=1). Samples were then resuspended in medium containing pronase (+) or pronase plus pronase inhibitor cocktail (−). After one hour at 4° C., sporozoites were lysed and CSP was immunoprecipitated and analyzed by SDS-PAGE and autoradiography. Panel E shows P. berghei sporozoites were dissected and purified in the absence (−) or presence (+) of E-64, washed, metabolically labeled, washed and either kept on ice (Time=0) or chased at 28° C. for two hours (Time=2). Sporozoites were then lysed and CSP was immunoprecipitated and analyzed by SDS-PAGE and autoradiography.

Immunofluorescence experiments with live sporozoites showed that the sporozoites were recognized by the N-terminal antiserum, suggesting that full-length CSP was on the surface (FIG. 3A). As shown, the majority of sporozoites had a uniform staining pattern; however, some parasites displayed a punctuate pattern. To confirm that full-length CSP was on the surface, sporozoites expressing green fluorescent protein (Natarajan, et al.) with sulfo-succinimidyl-6′-(biotinamido) hexanoate, a reagent that does not enter cells, were biotinylated. As shown in FIG. 3B, the high molecular weight form of CSP is biotinylated, indicating that it is found on the sporozoite surface. As a control, GFP, an intracellular protein, was immunoprecipitated and found that it was not labeled (FIG. 3C). These findings are in agreement with a previous study in which the high molecular weight form of CSP was found on the surface of Plasmodium vivax sporozoites (Gonzalez-Ceron, et al (1998)). If the high molecular weight CSP form is on the sporozoite surface, then this is the location of processing. However, other investigators found that the majority of CSP on the surface was the low molecular weight form, and concluded that processing occurred intracellularly (Yoshida, et al. (1981) and Cochrane, et al. (1982)). In these latter studies, CSP was immunoprecipitated from sporozoites that were metabolically-labeled and trypsinized. When compared with controls, trypsin-treated sporozoites were primarily missing the low molecular weight CS band, indicating that the high molecular weight CSP form was intracellular. In these experiments, however, trypsin was added immediately after labeling, which could not have allowed sufficient time for export of all the labeled CSP to the sporozoite surface. In order to investigate whether this was the case, the experiment was repeated and incorporated a one hour chase into the experimental design. Sporozoites were metabolically-labeled at 28° C. and then either kept on ice or chased at 28° C. for one hour; both in the presence of cyclohexamide to prevent further protein synthesis. Sporozoites were then treated with pronase or pronase plus an inhibitor cocktail. When the labeled parasites were not chased, the high molecular weight CSP form was not digested by pronase. However, if sporozoites were chased for one hour before pronase treatment, both CSP forms were digested, indicating that both forms are found on the sporozoite's surface and that this is the location of processing (FIG. 3D).

Sporozoites isolated from salivary glands of infected mosquitoes are invariably contaminated with mosquito debris. For this reason, it could not be determined whether the protease that cleaves CSP is of parasite or mosquito origin. To address this question, the kinetics of CSP processing in purified and unpurified sporozoites was compared and no difference was found between these groups. However, even purified sporozoites are associated with a small amount of mosquito debris. Therefore, sporozoites were dissected and purified in the presence of E-64, an irreversible inhibitor of cysteine proteases. After their isolation, sporozoites were washed and metabolically labeled in medium without E-64. Cysteine proteases of mosquito origin would be extracellular and therefore irreversibly inhibited by the E-64 present during sporozoite isolation. Sporozoites, however, continue to synthesize and/or secrete protease after the removal of E-64, allowing newly labeled CSP to be processed. As shown in FIG. 3E, CSP was processed with the same kinetics regardless of whether sporozoites were purified in the presence or absence of E-64. These data suggest that the CSP protease is of sporozoite origin.

### Example Three

### CSP Cleavage is Required for Cell Invasion

Proteolytic cleavage of cell surface and secreted proteins occurs during invasion of erythrocytes by the merozoite stage of Plasmodium (Blackman, et al. (2000). To determine whether CSP cleavage was required for sporozoite entry into cells, a variety of protease inhibitors were tested for their ability to inhibit sporozoite invasion of a hepatocyte cell line.

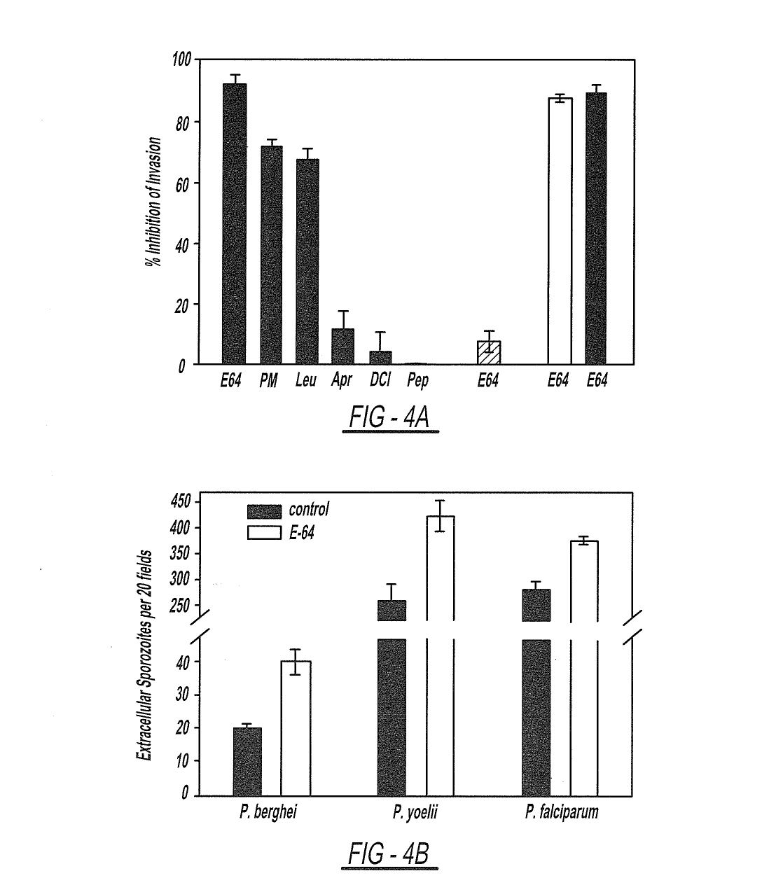

As set forth in FIG. 4, E-64 inhibits sporozoite invasion of, but not attachment to, cells. In Panel A of FIG. 4, the effect of protease inhibitors on invasion by Plasmodium sporozoites was shown. P. berghei (grey bars), P. yoelii (white bar) or P. falciparum (black bar) sporozoites were preincubated with the indicated protease inhibitors, added to cells and after one hour, the cells were washed, fixed and stained so that intracellular and extracellular sporozoites could be distinguished. A control (hatched bar) was performed in which the target cells were preincubated with E-64, the medium was removed and untreated P. berghei sporozoites were added as outlined above. Each point was performed in triplicate, 50 fields per well were counted and shown are the means with standard deviations. Inhibition of invasion was calculated based on the invasion rate for sporozoites pretreated with buffer alone, which was 54% for P. berghei, 26% for P. yoelii and 52% for P. falciparum (Abbreviations: PM, PMSF; Leu, leupeptin; Apr, aprotinin; DCI, 3,4 DCI; Pep, pepstatin). As for Panel B, it illustrates that attachment of sporozoites is enhanced in the presence of E-64. Shown are the numbers of extracellular sporozoites when sporozoites are preincubated with buffer alone (grey bars) or with E-64 (white bars). Data are from the invasion assay shown in Panel A.

The results, as set forth in FIG. 4, show that E-64 inhibited invasion by 90% and PMSF and leupeptin also had inhibitory activity. Pepstatin had no effect on invasion and the serine protease inhibitors aprotinin and DCI, which do not have activity against the papain-family cysteine proteases, also did not have significant inhibitory activity on invasion. To determine whether the effect of E-64 was on the sporozoite or the target cell, target cells were pretreated with E-64 and found that there was no inhibitory effect on sporozoite invasion (FIG. 4A). The ability of E-64 to inhibit sporozoite invasion was not restricted to P. berghei. Invasion by P. yoelii and P. falciparum sporozoites was also significantly inhibited by E-64 (FIG. 4A).

In the presence of E-64, the number of extracellular sporozoites was always enhanced, showing that there was an accumulation of attached sporozoites that were prevented from entering cells (FIG. 4B). Since attachment to cells is a distinct stage of sporozoite invasion (Pinzon-Ortiz, et al. (2001)), these results indicate that proteolytic cleavage of CSP is not required for this process. The inhibition of sporozoite invasion by E-64 provides evidence that CSP is cleaved during cell invasion. Therefore, intracellular sporozoites would not have full-length CSP on their surface. When it was tested whether intracellular sporozoites lost their reactivity to the N-terminal antiserum, however, the majority of sporozoites associated with cells had lost their reactivity to this antiserum regardless of whether they were intracellular or extracellular. In contrast, in the absence of cells, 80 to 90% of sporozoites stained with the N-terminal antiserum. These data suggested that cell contact was the trigger for CSP cleavage. To test this, sporozoites were preincubated with cytochalasin D (CD), an inhibitor of sporozoite invasion but not attachment to cells (Dobrowolki, et al. (1996) and Sinnis, et al. (1998)), in the presence or absence of E-64. They were then spun onto cells and brought to 37° C. for two minutes, fixed, and stained. As shown in Table 1, sporozoites incubated with CD plus E-64 stained with the N-terminal antiserum, while those incubated with CD alone did not. A control antibody, mAb 3D11, directed against the repeat region of CSP, bound to both E-64 treated and untreated sporozoites. Controls in which sporozoites were incubated without cells showed that neither elevated temperature nor serum alone had a significant effect on CSP cleavage (Table 1).

TABLE 1
Contact with Hepatocytes Triggers Cleavage of CSP

| | | | Method for | Number of |
|---|---|---|---|---|
| Exper- | | | Sporozoite | Sporozoites |
| iment. | Cells | Condition ^{a} | Visualization | Visualized ^{b} |
| 1 | Hepa 1-6 | CD | 3D11 | 244 ± 3 |
| | Hepa 1-6 | CD + E-64 | 3D11 | 230 ± 4 |
| | Hepa 1-6 | CD | α-N | 41 ± 1 |
| | Hepa 1-6 | CD + E-64 | α-N | 237 ± 5 |
| | no cells | control | α-N | 80% ± 0.4 |
| | no cells | CD | α-N | 85% ± 1.1 |
| | no cells | E-64 | α-N | 90% ± 4.1 |
| | no cells | CD + E-64 | α-N | 84% ± 0.5 |
| | no cells | CD + 10% serum | α-N | 80% ± 3.7 |
| 2 | Hepa 1-6 | CD | GFP | 452 ± 8 |
| | Hepa 1-6 | CD | α-N | 98 ± 2 |
| | Hepa 1-6 | CD + E-64 | GFP | 444 ± 6 |
| | Hepa 1-6 | CD + E-64 | α-N | 436 ± 8 |

^{a} P. berghei sporozoites (wildtype in experiment 1; GFP in experiment 2) were preincubated +/− E-64 and before addition to cover slips, CD was added to the indicated samples. Sporozoites were then spun onto cover slips, with or without cells as indicated, brought to 37° C. for two minutes, fixed, and stained with the indicated antisera.
^{b} Each point was plated in duplicate, 50 fields per cover slip were counted and shown are the means with standard deviations. When sporozoites were plated without cells, 100 to 200 sporozoites per cover slip were counted and shown is the percentage staining with the N-terminal antiserum.

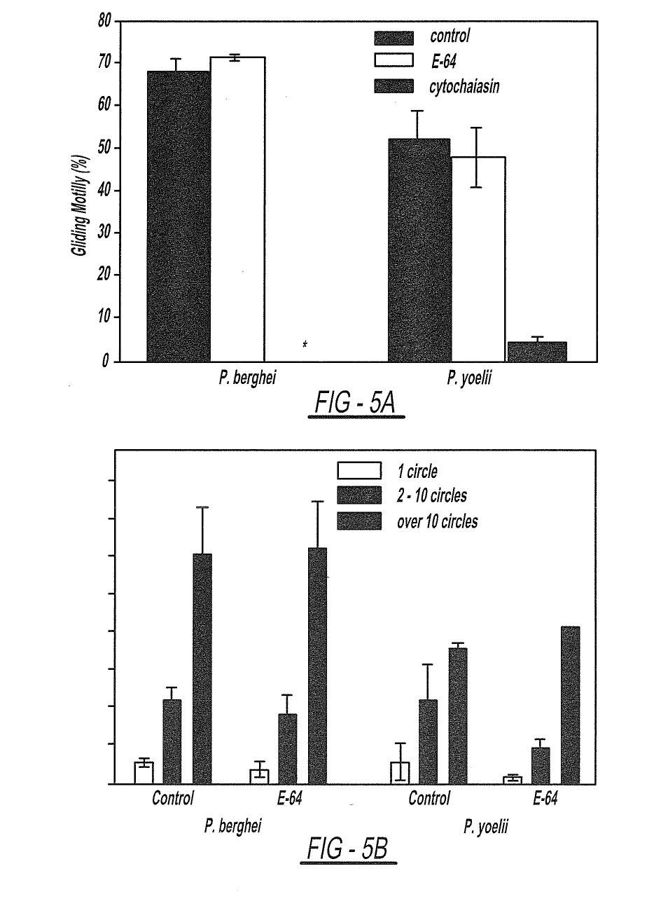

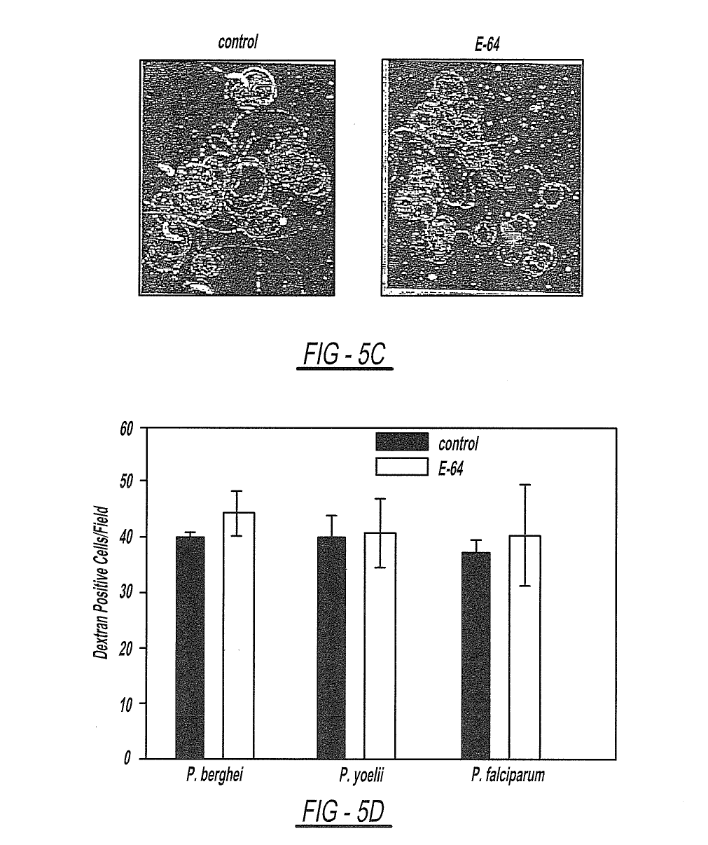

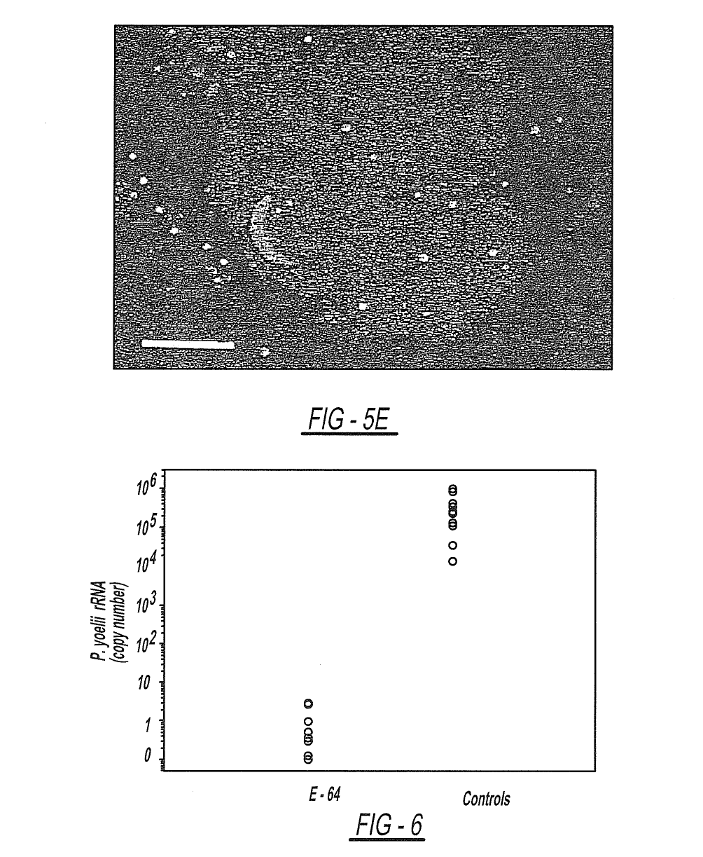

As set forth in FIG. 5, processing of CSP is not required for sporozoite motility or migration through cells. In Panels A-C of FIG. 5, sporozoites were preincubated with or without E-64, or with CD, and added to slides in the continued presence of the inhibitor. After one hour, trails were stained and counted. Each point was performed in triplicate, 100 sporozoites per well were counted and shown is the percentage of sporozoites associated with trails (panel A), the number of circles per trail for those sporozoites associated with trails (panel B) and a typical example of trails made by P. berghei sporozoites in the absence or presence of E-64 (panel C)(asterisk indicates that no trails were found). As for Panel D, sporozoites were preincubated with or without E-64 and added to cells with 1 mg/ml rhodamine-dextran. After one hour, cells were washed and the number of dextran positive cells per field was counted. Each point was performed in triplicate, 50 fields per cover slip were counted and shown are the means with standard deviations. Panel E demonstrates an intracellular sporozoite staining with the N-terminal antiserum in a dextran-positive cell (Bar=10 mm).

Since sporozoite motility is required for cell invasion (Sultan, et al. (1997)), it was tested whether CSP processing is required for motility. E-64 had no effect on the percentage of P. yoelii or P. berghei sporozoites that exhibited gliding motility (FIG. 5A). In addition, the average number of circles per gliding sporozoite was not different between treated and untreated sporozoites (FIGS. 5B and C). E-64 was tested to determine if it inhibited sporozoite migration through cells. Sporozoites migrate through several cells before productively invading a cell (Mota, et al. (2001)). The cell is wounded as the sporozoite passes through and if a high molecular weight fluorescent tracer is added to the medium, it enters wounded cells, which can then be quantified. E-64 did not inhibit sporozoite migration through cells (FIG. 5D).

Migrating sporozoites would retain full-length CSP on their surface. In order to test this, sporozoites with cells in the presence of dextran conjugated to rhodamine were incubated and then stained intracellular sporozoites with the N-terminal antiserum. Very few intracellular sporozoites reacting with the N-terminal antiserum were observed. However, the sporozoites that were recognized by this antiserum were always in dextran-positive cells, suggesting that they were migrating through these cells (FIG. 5E). Lastly, E-64 was tested as an inhibitor of malaria infection in vivo using a rodent model of the disease. Using a quantitative PCR assay, the amounts of parasite ribosomal RNA in the livers of mice, pretreated with E-64 or buffer, were compared and injected with 15,000 P. yoelii sporozoites.

As shown in FIG. 6, mice injected with E-64 were completely protected from malaria infection and proved that E-64 inhibits sporozoite infectivity in vivo. Mice were given three intraperitoneal injections of E-64 or buffer alone at 16 hours, 2.5 hours, and 1 hour prior to intravenous inoculation of P. yoelii sporozoites. Forty hours later, the mice were sacrificed, total liver RNA was extracted and malaria infection was quantified by reverse transcription followed by real time PCR using primers specific for P. yoelii 18S rRNA. A standard curve was generated using a plasmid containing the P. yoelii 18S gene and infection is expressed as the number of copies of the 18S rRNA. Shown are the results of two experiments. There were six mice per group in each experiment.

### Example Four

### Inhibition of CSP Cleavage

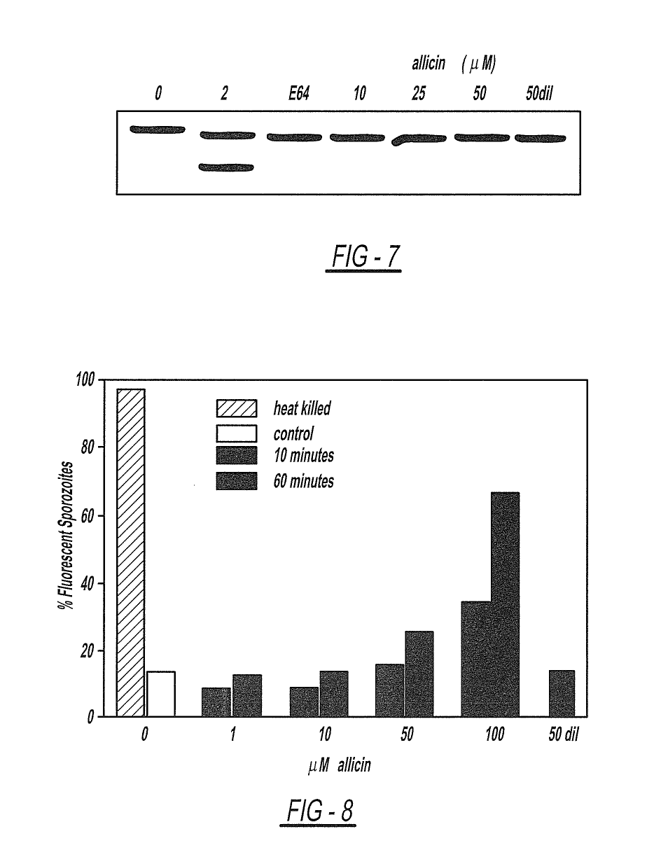

As set forth in FIG. 7, it is shown that allicin prevents cleavage of CSP. P. berghei sporozoites were metabolically labeled with [³⁵S]Cys/Met and kept on ice (lane 1) or chased for two hours in the absence of protease inhibitors (lane 2), in the presence of 10 μM E-64 (lane 3), or in the presence of the indicated concentrations of allicin (lanes 4-6). Lane 7 represents labeled sporozoites chased in the presence of 50 μM allicin for 10 minutes, which was then diluted to 4.2 μM for the remainder of the chase. After two hours, the parasites were lysed, CSP was immunoprecipitated and analyzed by SDS-PAGE, and autoradiography.

It has been previously shown that the cysteine protease inhibitor E-64 prevents proteolytic cleavage of the major surface protein of sporozoites, the circumsporozoite protein (CSP) (Coppi, A.). Further, allicin has been shown to react with free sulfhydryl groups (Rabinkov, A.), and in this way can reversibly inhibit cysteine proteases (Ankri, S.). Pulse chase metabolic labeling experiments in the presence of allicin indicate that CSP cleavage is inhibited by 10, 25, and 50 μM allicin (FIG. 7). The degree of inhibition was comparable to that observed with E-64. In addition, chasing with 50 μM allicin for 10 minutes followed by dilution to 4.2 μM for the remainder of the chase, prevented CSP cleavage to the same extent as when 50 μM allicin was present during the entire chase.

### Example Five

### Allicin Toxicity

As set forth in FIG. 8, toxicity of allicin on Plasmodium sporozoites was demonstrated. P. berghei sporozoites were incubated with the indicated concentrations of allicin for 10 minutes (grey bars) or 60 minutes (black bars) before the addition of propidium iodide. The “50 dil” bar indicates that sporozoites were incubated with 50 μM allicin for 10 minutes, followed by 50 minutes of incubation in 4.2 μM allicin. Control sporozoites were incubated in the absence of allicin for 60 minutes (white bar) or were heat killed (diagonally striped bar). For each sample, 200 sporozoites were counted and the percentage staining with propidium iodide is shown.

In order to determine if the effect of allicin on CSP cleavage was due to a toxic effect on the sporozoites, parasites were incubated for ten minutes or one hour with different concentrations of allicin and then propidium iodide was added. Propidium iodide is a dye that is excluded by viable cells but penetrates the cell membranes of dying or dead cells. When sporozoites were incubated with either 1 or 10 μM allicin for up to one hour, the percentage of sporozoites that took up the dye was no different from the untreated control (FIG. 8). A ten minute incubation with 50 μM allicin also did not kill sporozoites; however, when the incubation time was increased to one hour, the number of sporozoites taking up the dye increased 1.5-fold, indicating that longer exposures to 50 μM allicin had some toxic effects on the sporozoites. Treatment of sporozoites with 50 μM allicin for 10 minutes, followed by dilution of the allicin to 4.2 μM and an additional 50 minute incubation did not increase the number of fluorescent sporozoites compared to the untreated control. At concentrations higher than 50 μM, allicin was toxic to the sporozoites even after only ten minutes of exposure.

### Example Six

### Effects of Allicin on Preventing Gliding Motility

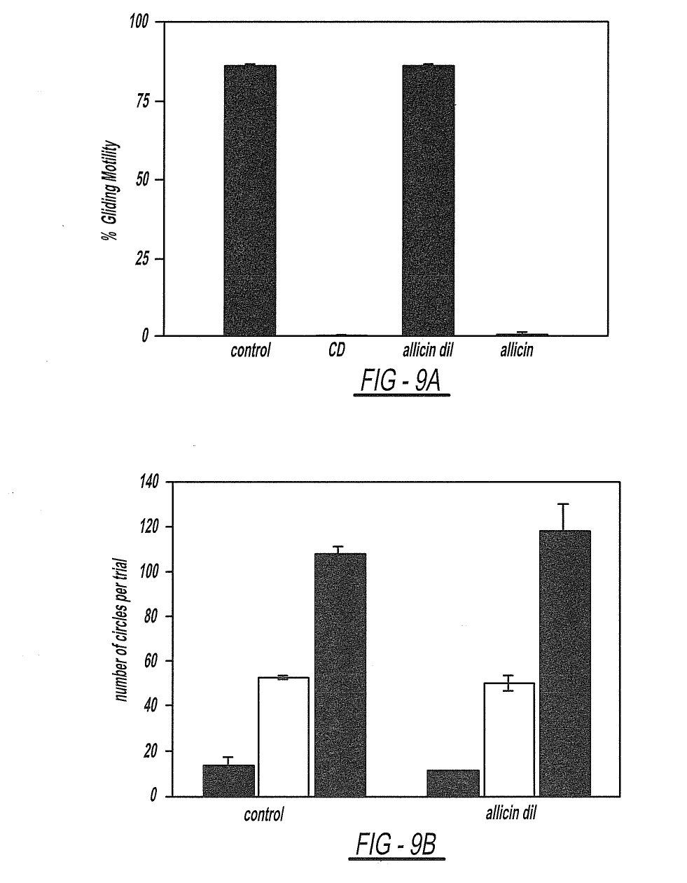

As set forth in FIG. 9, the effect of allicin on gliding motility was shown. P. berghei sporozoites were preincubated in buffer alone, 1 μM cytochalasin D, or 50 μM allicin and then added to wells for one hour at 37° C. after which gliding motility was quantified. The sporozoites pretreated with allicin were either kept in 50 μM allicin during the motility assay (50) or diluted 12-fold so that the final concentration of allicin was 4.2 μM (50 dil). Shown is (A) the percentage of sporozoites that exhibited gliding motility and (B) the number of gliding sporozoites exhibiting 1 (black bars), 2-10 (light grey bars), or >10 (dark grey bars) circles per trail. Each point was performed in triplicate, 200 sporozoites/well were counted, and the means±SD are shown.

Since uptake of propidium iodide is a terminal event, it was determined whether sporozoites incubated with allicin were still motile. Plasmodium sporozoites exhibit a unique form of substrate-dependent locomotion, termed gliding motility, which is required for cell invasion (Sultan, A., et al.). If sporozoites were motile in the presence of allicin, then the compound was not affecting the overall metabolic activity of the parasites. In motility assays, allicin was tested and found that preincubation with 50 μM allicin for ten minutes followed by dilution to 4.2 μM had no effect on gliding motility (FIG. 9A). In addition, both the percentage of sporozoites that exhibited gliding motility as well as the number of circles per trail were the same in the allicin-treated sporozoites compared to controls (FIG. 9B). However, if allicin was not diluted and sporozoites were kept in 50 μM allicin for the duration of the assay, gliding motility was completely inhibited (FIG. 9A). This is consistent with the toxicity profile of allicin that was observed using propidium iodide: prolonged incubations in 50 μM allicin are toxic, whereas a ten minute incubation in 50 μM allicin followed by an incubation in 4.2 μM is not. As a result, it has been shown that allicin does not prevent gliding motility.

### Example Seven

### Effect of Allicin on Cell Invasion

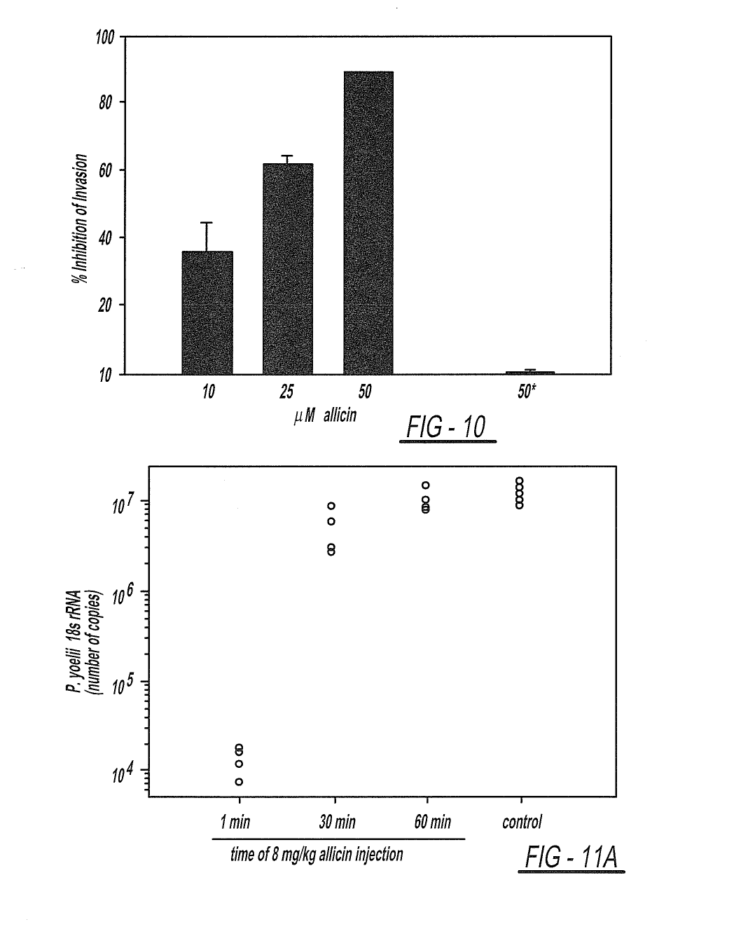

According to FIG. 10, it was demonstrated that allicin inhibits sporozoite invasion of host cells. P. berghei sporozoites were pretreated with the indicated concentrations of allicin for ten minutes and then diluted 12-fold and added to cells for one hour. Cells were then fixed, stained, and the number of intracellular and extracellular sporozoites were counted. “50*” indicates that Hepa 1-6 cells were preincubated with 50 μM allicin for one hour, washed, and untreated sporozoites were then added to the cells. Each point was performed in triplicate, ≧50 fields/well were counted, and the means±SD are shown. Inhibition of invasion was calculated based on the invasion rate for sporozoites pretreated with buffer alone which was 57%.

Since allicin inhibited CSP cleavage and previous studies showed that cleavage is associated with cell invasion, it was tested whether allicin would inhibit invasion of host cells. For these experiments and as set forth above, P. berghei sporozoites were pretreated with 10, 25, and 50 μM allicin for 10 minutes, diluted 12-fold and added to host cells. As shown in FIG. 10, allicin inhibited sporozoite invasion of cells in a dose dependent manner. At the lowest concentration tested (10 μM), allicin inhibited invasion by 37% compared to the untreated control. When sporozoites were pretreated with 50 μM allicin, invasion was inhibited by 89%, a result similar to that seen when sporozoites are pretreated with E-64 (Coppi, A., et al.). Importantly, pretreatment of host cells with 50 μM allicin had no effect on invasion (FIG. 10). Allicin thus prevents cell invasion.

### Example Eight

### Inhibition of In Vivo Sporozoite Infectivity

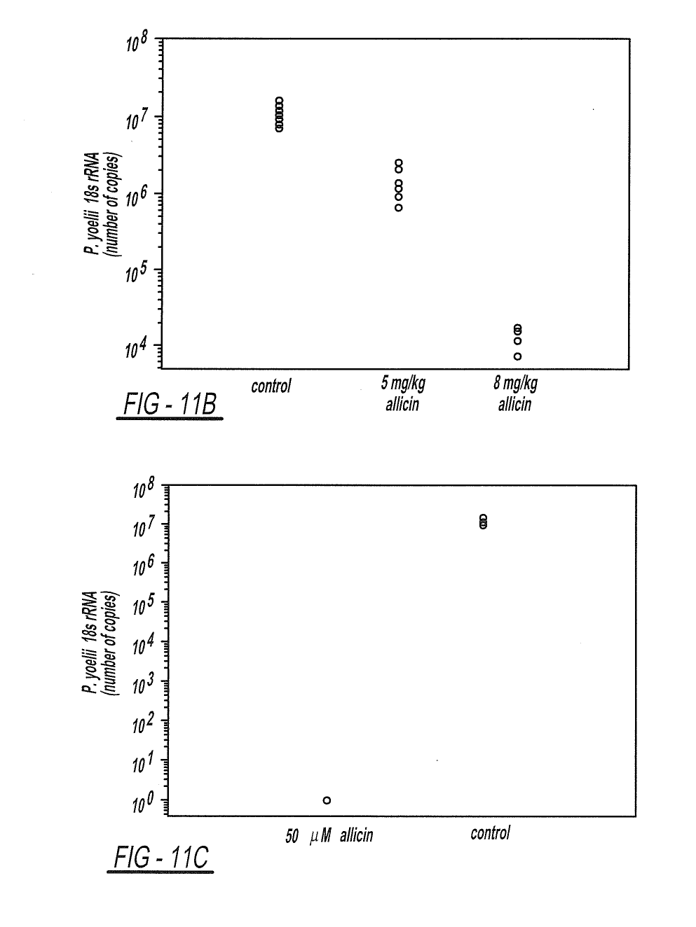

As set forth in FIG. 11, allicin decreases sporozoite infectivity in vivo. Mice were injected with allicin or buffer alone before injection of P. yoelii sporozoites. Forty hours later, mice were sacrificed, total liver RNA was extracted, and malaria infection was determined by quantitative PCR. Infection is expressed as the number of copies of P. yoelii 18S rRNA. FIG. 11A shows that mice were injected intravenously with 8 mg/kg allicin 1 minute, 30 minutes, and 60 minutes before injection of sporozoites, while FIG. 11B shows mice were injected intravenously with 5 mg/kg allicin, 8 mg/kg allicin, or buffer alone one minute before injection of sporozoites. Finally, FIG. 11C shows sporozoites were preincubated with 50 μM allicin for ten minutes, diluted 12-fold with buffer, and injected into mice (n=6 mice per group).

Allicin was tested to determine its ability to inhibit sporozoite infectivity in vivo using the rodent malaria parasite P. yoelii. Mice were injected with allicin or buffer alone at different times before injection of sporozoites. Forty hours after sporozoite injection, the parasite burden in the liver was determined by RT followed by real time PCR. As shown in FIG. 11A, mice injected with allicin had decreased levels of infection and inhibition of infection was correlated with the length of time between allicin injection and sporozoite injection. When allicin was administered just before injection of sporozoites, it significantly decreased infectivity compared to untreated controls (P<0.001). Allicin injected thirty minutes prior to injection of sporozoites also resulted in decreased infectivity compared to the untreated mice (P<0.001), but the protective effect was not as great as that seen when allicin was administered just before sporozoite injection. Administration of allicin one hour prior to sporozoite injection yielded little protection (P<0.25). The experiment found that protection was dose-dependent. A dose of 8 mg/kg resulted in a 1000-fold decrease in infection compared to controls (P<0.001), whereas a dose of 5 mg/ml resulted in a 10-fold reduction in infection (P<0.001) (FIG. 10B).

The decrease in efficacy of allicin over time is a consequence of its rapid decomposition in vivo (Brodnitz, M. H., et al.). In order to test the inhibitory activity of allicin in vivo, before its catabolism in the blood, a second set of experiments were performed in which mice were injected with P. yoelii sporozoites preincubated with 50 μM allicin or buffer alone. As shown in FIG. 10C, mice injected with the allicin pretreated sporozoites showed no evidence of malaria infection.

### Example 9

### Rationale for Creation of Region I Deletion Mutant

CS proteins from all species of Plasmodium have a similar overall structure, comprised of a central species-specific repeat region and 2 highly conserved domains; a 5 amino acid sequence, called region I, found just before the repeats and a C-terminal cell adhesive motif with similarity to the type I thrombospondin repeat (TSR; FIG. 12A; reviewed in (Sinnis and Nardin, 2002)). Applicants' previous studies have shown that polyclonal antisera generated to a peptide encompassing the entire NH₂-terminal third of CSP does not recognize the processed lower MW form of the protein (Coppi et al., 2005). Monoclonal antibodies to the repeat region, however, recognized both full-length and cleaved forms of the protein (Coppi et al., 2005). Together these data show that the cleavage site is located between the distal portion of the NH₂ terminus and the CSP repeats. Since the highly conserved region I is located between the NH₂ terminus and the repeats, Applicants investigated whether this 5 amino acid sequence contained the cleavage site by generating mutant parasites that lacked region I.

### Example 10

### Generation and Verification of Region I Deletion Mutant

Sporozoites expressing CSP in which region I was deleted (ΔRI) and recombinant control sporozoites (RCon) into which a wild type CSP gene was transfected, were generated by double homologous recombination (FIG. 12B). CSP lacking region I was made using a PCR-based strategy (see supplementary materials and methods in Example 19). To direct homologous recombination to the correct locus, transfection plasmids were created containing the CSP gene, either wild type or lacking region I, with its 5′ and 3′ promoter elements, the selection cassette and additional CSP 5′UTR (FIG. 12B). Transfection of blood stage P. berghei schizonts was performed as previously outlined and transfectants were selected with pyrimethamine and cloned in mice (Menard and Janse, 1997). Clones were checked for integration of the construct into the correct locus by PCR and Southern Blot (FIG. 12C). ΔRI clones were checked for the maintenance of the deletion by sequencing the PCR product produced by primers P3 and P4 (FIG. 12C; sequence data not shown). Western blot analysis (FIG. 14A) and immunofluorescence studies (FIG. 12D) showed that CSP was expressed in normal amounts in ΔRI sporozoites and it was exported to the parasite surface similar to wild type sporozoites.

### Example 11

### CSP Processing is Dramatically Decreased in Region I Deletion Mutants

It was first determined whether ΔRI sporozoites processed CSP normally. Western blot analysis of ΔRI and RCon sporozoite lysates showed that only small amounts of CSP were processed in the ΔRI parasites (FIG. 13A). To study this in more detail we performed pulse-chase metabolic labeling experiments, in the absence and presence of hepatocytes.

CSP processing occurs on the sporozoite surface and is a significantly more rapid process in the presence of hepatocytes suggesting that contact with target cells triggers secretion of the responsible protease (Coppi et al., 2007). In the absence of cells, the half-life of full-length protein after export is between 1 to 2 hrs (Coppi et al., 2005). Pulse-chase metabolic labeling experiments performed with ΔRI parasites in the absence of cells showed that these mutant parasites did not process any of the labeled protein in the 4 hour chase whereas RCon parasites cleaved CSP with expected kinetics (FIG. 13B). To test the effect of hepatocytes on the kinetics of CSP cleavage metabolically-labeled RCon and ΔRI sporozoites were chased for 1 hr so that labeled CSP had time to be exported to the sporozoite surface. When labeled RCon sporozoites were then added to cells cleavage went to completion within 5 minutes (FIG. 13C). In contrast, ΔRI sporozoites processed approximately 30% of the labeled CSP after their addition to hepatocytes, and with significantly slower kinetics compared to RCon parasites (FIG. 13C).

### Example 12

### Region I Deletion Mutants in the Mosquito Vector

Region I has been hypothesized to play a role in salivary gland invasion (Mo Myung et al., 2004). It was therefore assessed whether a reduced ability to cleave CS played a role in parasite infectivity in the mosquito vector. Erythrocytic stages of recombinant parasites, which grew normally, were used to feed Anopheles stephensi mosquitoes. At different time points after feeding, mosquitoes were dissected and sporozoites in oocysts, hemolymph and salivary glands were enumerated in ΔRI, RCon and wild type untransfected parasites. Deletion of region I had no effect on sporozoite development in oocysts (FIG. 14A) nor on sporozoite egress from oocysts into the hemolymph (FIG. 14B). However we consistently found that ΔRI parasites invaded salivary glands with a 10 to 15% lower efficiency compared to RCon or wild type parasites. Because of the inherent variability in salivary gland invasion by different batches of sporozoites, we repeated the cycle six times and found that this difference was reproducible, suggesting that these parasites were marginally less invasive for salivary glands. To verify that salivary gland sporozoites had entered and were not just stuck to the outside of the glands, we counted the number of sporozoites associated with the glands before and after trypsinization. There was no difference in the percentage of sporozoites found on the outside of the glands when salivary glands harboring ΔRI, wild type and RCon sporozoites were compared (Table 2), indicating that the ΔRI sporozoites were able to enter salivary glands albeit in lower overall numbers.

TABLE 2
Localization of wild type, RCon and
ΔRI salivary gland sporozoites.

| | Wild type | | RCon | | ΔRI | |
|---|---|---|---|---|---|---|
| Day | Inside | Outside | Inside | Outside | Inside | Outside |
| 18 | 80.1% | 19.9% | 79.3% | 20.7% | 76.6% | 23.4% |
| 19 | 72.9% | 20.8% | 80.8% | 19.2% | 78.5% | 21.5% |
| 20 | 79.0% | 21.0% | 80.0% | 20.0% | 78.8% | 21.2% |

### Example 13

### Infectivity to Hepatocytes In Vitro of the Region I Deletion Mutant

It was next investigated whether the altered CSP processing in ΔRI sporozoites played a role in hepatic host cell invasion by assaying the ability of our mutant to invade a hepatocyte cell line, Hepa 1-6 in vitro. Sporozoites were incubated with cells for 1 hour and then stained so that intracellular and extracellular parasites could be distinguished and counted. No difference in the number of sporozoites that attached to Hepa 1-6 cells (FIG. 15A) were observed. However, ΔRI sporozoites were significantly impaired in their ability to invade cells (FIG. 15B).

Not all intracellular sporozoites develop into EEF as some intracellular parasites will be in the process of migrating through the cell (Mota et al., 2001). Because reliable markers for the early parasitophorus vacuole (PV) required for EEF development are lacking, it is difficult to distinguish, at early time points, sporozoites that have productively invaded from those that are migrating through. To address this issue, invasion assays were performed in the presence of E-64 which inhibits productive invasion of cells and therefore allows the approximation of the number of intracellular parasites that are in the process of migrating (Coppi et al., 2005). As shown in FIG. 15B, there are few intracellular wild type and RCon sporozoites in the presence of E-64 indicating that the majority have productively invaded. There are similarly low numbers of intracellular ΔRI parasites in the presence of E-64, however because the overall invasion rate is low, these constitute between 30 to 40% of the total number of intracellular sporozoites. These findings suggest that the rate at which ΔRI sporozoites productively invade hepatocytes in vitro is approximately 6-fold lower than wild type. Consistent with the decrease observed in the invasion assay, there was a 6-fold decrease in the number of ΔRI sporozoites that developed into mature EEFs in vitro when compared to wild type and RCon parasites (FIG. 15C).

### Example 14

### In Vivo Infectivity of the Region I Deletion Mutant

Next, the infectivity of ΔRI sporozoites in C57BI/6 mice and outbred Swiss Webster mice was investigated. Sporozoites were injected intravenously and parasite liver load was determined 40 hours later by quantitative RT-PCR. Using this methodology, only sporozoites which have invaded hepatocytes and undergone many cycles of replication are detectable since the small amount of rRNA present in the injected sporozoites or in early liver stages is below the sensitivity of the assay (Briones et al., 1996). As shown in FIGS. 16A and 16B, infectivity of the ΔRI sporozoites is decreased by 15-fold in C57BI/6 mice and by 10-fold in Swiss Webster mice.

In order to determine whether the route of administration had an effect on infectivity of ΔRI sporozoites, Applicants tested infectivity after intravenous and intradermal inoculation. Mosquitoes inject sporozoites into the dermis of the mammalian host (Matsuoka et al., 2002; Medica and Sinnis, 2005) and recent evidence indicates that exit from the skin and entry into the blood stream requires that sporozoites traverse cell barriers in the dermis (Amino et al.; Bhanot et al., 2005). Comparison of sporozoite infectivity after intravenous and intradermal injection therefore additionally tests whether the ΔRI sporozoites are competent in exiting the dermis. In these experiments we determined the pre-patent period, or time to blood stage infection as detected by Giemsa stained blood smears. When the sporozoite inoculum and resulting liver stage burden is large, erythrocytic stage parasites can be observed starting from day 3 post-sporozoite inoculation. A one day delay in patency is considered to indicate a 5 to 10-fold decrease in liver stage parasite burden (Gantt et al., 1998). When ΔRI sporozoites were injected into mice there was found a 2 day delay in pre-patent period compared to controls. These results correlate well with the 15-fold decrease in parasite load observed with the RT-PCR assay (Table 3 and FIGS. 16A and 16B). In addition, the infectivity of ΔRI sporozoites did not depend on their route of administration, suggesting that cleavage of CSP is important for hepatocyte invasion and is not involved in exit from the dermis and localization to the liver.

TABLE 3
Prepatent period at mice inoculated
with control or mutant sporozoites.

| | | Number of Mice | Prepatent |
|---|---|---|---|
| Parasite | Route of Inoculation* | Positive | Period |
| RCon | IV | 5/5 | 3.0 |
| ΔRI | IV | 5/5 | 5.0 |
| RCon | ID | 5/5 | 3.0 |
| ΔRI | ID | 5/5 | 5.0 |

*5000 sporozoites were inoculated intravenously (IV) or intradermally (ID) into Swiss Webster mice.

### Example 15

### Motility and Migration of the Region I Deletion Mutant

Similar to other invasive stages of Apicomplexan parasites, Plasmodium sporozoites exhibit a substrate-dependent type of locomotion called gliding motility (reviewed in (Kappe et al., 2004)). Invasion by Apicomplexan zoites is an active process and zoites must be motile in order to invade cells.

Because of their defect in invasion, it was tested whether ΔRI mutants exhibited normal gliding motility. On glass surfaces, sporozoites tend to glide in circles and enumeration of the number of circles left behind by each sporozoite is an indication of the extent to which they glide. As shown in FIGS. 17A-17C, ΔRI mutants exhibited normal gliding motility, similar to that observed with wild type and RCon sporozoites. ΔRI sporozoites have a larger proportion of trails that contain over 10 circles, suggesting that they have enhanced motility compared to controls (FIG. 17B).

Later, the cell traversal activity of ΔRI sporozoites was observed. Previous studies have shown that sporozoites can interact with cells in one of two ways: they can productively invade a cell, forming a parasitophorous vacuole in which they will replicate, or they can migrate through a cell, breaching the cell's plasma membrane in the process (Mota et al., 2001). The ability to traverse cell barriers likely enables sporozoites to reach the liver from their injection site in the dermis (Amino et al.; Coppi et al., 2007). Applicants had previously found that CSP cleavage was specifically associated with productive invasion of hepatocytes (Coppi et al., 2005) and were therefore interested to compare the migratory activity of the ΔRI with wild type and RCon sporozoites. As shown in FIG. 17C, ΔRI parasites exhibited enhanced migratory activity compared to wild type or RCon sporozoites. In the presence of E-64, a cysteine protease inhibitor that inhibits productive invasion, sporozoites display 4 to 5-fold increased migratory activity and the migratory activity of ΔRI mutants is similar to wild type sporozoites treated with E-64 (FIG. 17C).

### Example 16

### Function of CSP Cleavage During Sporozoite Invasion of Hepatocytes

Proteolytic cleavage of CSP is required for invasion, however, its precise role in this process is not known. Previous data demonstrate that CSP binds to highly sulfated heparan sulfate proteoglycans and this binding is responsible, at least in part, for sporozoite localization to the liver [reviewed in (Sinnis and Coppi, 2007)]. CSP has two heparin-binding domains, one in the NH₂-terminus and another in the TSR, located in the carboxy terminus of CSP (Rathore et al., 2002; Sinnis et al., 1994). Our findings raise the possibility that initial binding of the NH₂-terminus to HSPGs leads to cleavage and conformation change of the protein which then exposes a high affinity HSPG-binding site, namely the TSR.

To test whether the conformation of native CSP changes following cleavage, Applicants stained WT sporozoites with polyclonal antisera raised against peptides containing the entire NH₂-terminus or COOH-terminus of CSP (peptides shown in FIG. 12A) before and after contact with hepatocytes since we had previously shown that contact with hepatocytes triggers CSP cleavage [(Coppi et al., 2005) and FIG. 13]. Before contacting hepatocytes, WT sporozoites stain with antiserum to the NH₂-terminus but not with antiserum to the COOH-terminus of CSP (FIG. 18A). In the presence of hepatocytes, however, CSP is cleaved and the majority of WT sporozoites quickly loose their reactivity to the anti-NH₂ serum and gain reactivity to the anti-COOH serum (FIG. 18B). In contrast, when assayed at multiple time points, the majority of ΔRI sporozoites never become positive for staining with antiserum recognizing the COOH-terminus of CSP (FIGS. 18A-18B). Since proteolytic processing of CSP is significantly reduced in ΔRI sporozoites, these data suggest that CSP cleavage alters protein conformation and exposes the COOH-terminus, containing the cell-adhesive TSR, during hepatocyte invasion.

### Example 17

### Antibodies to CSP Function by Inhibiting Proteolytic Processing

Because inhibition of CSP processing may represent a means by which sporozoite infectivity could be diminished, we tested whether antiserum to the NH₂-terminus of CSP might recognize the cleavage site and decrease sporozoite infectivity. In addition, based on our finding that the cleavage site is immediately adjacent to the repeat region, and the fact that antibodies to the repeat region of CSP are effective in inhibiting sporozoite infectivity (Hollingdale et al., 1984; Hollingdale et al., 1982), Applicants also tested whether anti-repeat antibodies might inhibit CSP processing.

To test the activity of these antisera in our cleavage assay, we performed pulse-chase metabolic labeling experiments in which we added anti-repeat mAbs or anti-NH₂ antiserum to sporozoites during the chase. Polyclonal antisera to the NH₂-terminal third of P. berghei CSP did not have any effect on cleavage (FIG. 19A, left panel). However, mAb 3D11, directed against the repeat region of P. berghei inhibited cleavage of P. berghei CSP in a dose-dependent manner: 100 μg/ml completely inhibited processing, 10 μg/ml inhibited cleavage by approximately 50 to 75% and 1 μg/ml of antibody had little inhibitory activity (FIG. 19A, left panel).

Inhibition of hepatocyte invasion by mAb 3D11 paralleled the inhibitory activity of the antibody on CSP cleavage (FIG. 19A, right panel). As a control Applicants used 100 μg/ml of mAb 2A10, directed against the repeat region of P. falciparum CSP and observed no effect on P. berghei CSP cleavage or sporozoite invasion.

Since CSP processing occurs in all Plasmodium species [reviewed in (Coppi et al., 2005)] and the overall structure of CSP is conserved across species, we performed similar experiments with sporozoites of the human malaria parasite, P. falciparum. MAb 2A10, directed against the repeat region of P. falciparum CSP, inhibited cleavage in a dose-dependent fashion. The inhibitory activity of the antibody on hepatocyte invasion paralleled its effect on CSP cleavage (FIG. 19B). MAb 3D11, directed against the P. berghei repeat region had no effect on cleavage of P. falciparum CSP or P. falciparum sporozoite invasion.

### Example 18

### Antibodies Targeting the Minor Repeats of the Circumsporozoite Protein Effectively Inhibit CSP Cleavage and Sporozoite Infectivity

Shown in FIG. 20A is a schematic of CSP of the human malaria parasite, Plasmodium falciparum with the portions of the protein recognized by mAb 2A10 and mAb 2B6 indicated. Region I is the proteolytic cleavage site and the TSR is the cell-adhesive motif with similarity to the type I thrombospondin repeat that is found in the COOH-terminus of the protein. FIG. 20C is a pulse-chase metabolic labeling experiment in which P. falciparum sporozoites were metabolically labeled with ³⁵S-Cys/Met for 1 hour and then placed on ice (0 hours) or chased for 2 hours in the absence (2 hour) or presence of the indicated concentrations of monoclonal antibodies directed against the repeat region (mAbs 2A10 and 2B6). As shown, mAb 2A10 effectively inhibits cleavage at 100 micrograms/ml and partially inhibits cleavage at 10 micrograms/ml. In contrast, mAb 2B6 which specifically targets the minor repeats, effectively inhibits cleavage at 10 micrograms/ml and partially inhibits cleavage at 1 microgram/ml. When tested in sporozoite invasion assays, the effect of each monoclonal antibody parallels their effect on CSP cleavage (FIG. 20B).

### Example 19

### Supplementary Materials and Methods Construction of Plasmid pCSRep containing wild type or Region I-deleted CSP

pCSComp (Thathy et al., 2002) which contains a drug selection cassette consisting of a copy of the human DHFR gene flanked by 2.2 kb of 5′UTR and 0.55 kb of 3′UTR of P. berghei DHFR-TS followed by a CSP cassette (Eichinger et al., 1986) consisting of a WT copy of the P. berghei CSP gene flanked by 1.3 kb of CSP 5′UTR and 450 bp of CSP 3′UTR was used to build pCSRep. In order generate a targeting construct that would replace the endogenous CSP locus pCSComp required additional CSP 5′UTR to be placed upstream of the selection cassette. This was obtained from p9.5ΔE (Thathy et al., 2002) between the EcoRV and XbaI sites, using forward primer (5′-GTGCTCGAGTAAT ATATGAAAATAATGAATGAGG-3′; introduced XhoI site, underlined) and reverse primer (5′-CTCGTCGACAATAAATTGGTTTATGAAATTAGC-3; introduced HinclI site, underlined). The resulting 730 bp PCR product was cloned into pCR4-TOPO (Invitrogen) and terminal restriction sites were then added for cloning into pCSComp using forward primer (5′-AAACTGCAGCTCGAGTAATATATGAAAATAATGAATG-3′) which introduced a new PstI site immediately before the existing XhoI site and reverse primer (5′-AAAACTGCAGACAATAAATTGGTTTATGAAATTAGC-3′) which introduced another PstI site, underlined. The PCR product was cloned, its sequence verified and then cloned into the PstI site at the 5′ end of the selection cassette in pCSComp. This final construction was called pCSRep and contained wild type CSP.

In order to generate CSP in which region I was deleted, plasmid p9.5ΔXΔIS was used as the source for the P. berghei CSP gene (Eichinger et al., 1986) and region I was deleted as follows (FIG. 21): A 128 bp fragment from the CSP open reading frame was amplified by PCR using forward primer ΔRI-P1 (5′-GTATCACGTGC TTAACTCTAAG-3′; existing Pml1 site underlined) and ΔRI-P2 (5′-GCAATATTATTACG CTCTATTTTTTCG-3′; introduced SspI site, underlined). A 776 bp fragment from the CSP was also amplified using forward primer ΔRI-P3 (5′-GAGCGTAATAATAAATTG AAACAAAGGCCTCCACCACCAAACCC-3′; introduced Stul site underlined) and reverse primer ΔRI-P4 (5′-GTTTATTTAATTAAAGAATACTAATAC-3′; existing PacI site, underlined). Both PCR products were amplified with Taq polymerase using the following cycling conditions: 94° C. 1 minute followed by 30 cycles of (94° C. 30 seconds, 53° C. 30 seconds, 72° C. 1 minute), and ending with 72° C. 5 minutes. Following this they were gel purified using the QIAquick gel extraction kit (Qiagen, Valencia, Calif.) and then the 128 bp fragment was digested with PmlI and SspI and the 776 bp fragment was digested with Stul and PacI. The digested fragments now lacked the nucleotide sequences representing region I and they were ligated overnight at 14° C. in the presence of T4 DNA ligase. Since several ligation products were possible and the correct Pml-Pac fragment was only of interest, a PCR amplification was performed of the ligation reaction with primers ΔRI-P1 and ΔRI-P4 using the following cycling conditions: 94° C. 1 minute followed by 35 cycles of (94° C. 30 seconds, 57° C. for 30 seconds and 72° C. for 1 minute) and ending with 72° C. 5 minutes. The resulting 873 PmlI-PacI fragment was cloned into pCR4-TOPO vector and several plasmid clones were sequenced with to verify that region I was deleted and the repeat regions intact. The CSP PmlI-PacI fragment with region I-deleted was then used to replace the endogenous PmlI-PacI fragment in pCSComp (Thathy et al., 2002).

The above Examples demonstrate that CSP is cleaved in region I by a cysteine protease which allows malarial infection to take place. Inhibition of this cleavage can be accomplished by administering a cysteine protease inhibitor or by administering an inhibitor that inhibits a productive association between the protease and its substrate, CSP. Thus malaria infection can be prevented before it has entered an infectious stage, i.e. malaria can be targeted at the sporozoite stage instead of waiting for infection to occur and treating at the erythrocytic stage. Importantly, lower levels of antibodies that target the region adjacent to the cleavage site have been shown to be effective.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings It is, therefore, to be understood that within the scope of the described invention, the invention can be practiced otherwise than as specifically described.

### REFERENCES

Amino, R., Giovannini, D., Thiberge, S., Gueirard, P. Dubremetz, J., Prévost, M., Ishino, T., Yuda, M., and Ménard, R. Host cell traversal is important for progression of the malaria parasite from the dermis to the liver. J Exp Med. In Press.
Ankri, S., and D. Mirelman. 1999. Antimicrobial properties of allicin from garlic. Microbes and Infection 1:125-129.
Ankri, S., T. Miron, A. Rabinkov, M. Wilchek, and D. Mirelman. 1997. Allicin from garlic strongly inhibits cysteine proteinases and cytopathic effects of Entamoeba histolytica. Antimicrob. Agents Chemother. 41:2286-2288.
Bhanot, P., Schauer, K., Coppens, I., and Nussenzweig, V. (2005). A surface phospholipase is involved in the migration of Plasmodium sporozoites through cells. J Biol Chem 280, 6752-6760.
Blackman, M. J. 2000. Proteases involved in erythrocyte invasion by the malaria parasite: function and potential as chemotherapeutic targets. Curr. Drug Targets 1:59-83.
Breman, J. G., M. S. Alilio, and A. Mills. 2004. Conquering the intolerable burden of malaria: what's new, what's needed: a summary. Am. J. Trop. Med. Hyg. 71:1-15.
Briones, M. R. S., Tsuji, M., and Nussenzweig, V. (1996). The large difference in infectivity for mice of Plasmodium berghei and Plasmodium yoelii sporozoites cannot be correlated with their ability to enter into hepatocytes. Mol Biochem Parasitol 77, 7-17.
Brodnitz, M. H., J. V. Pascale, and L. V. Derslice. 1971. Flavour components of garlic extract. J. Agric. Food Chem. 19:273-275.
Bruna-Romero, O., J. C. R. Hafalla, G. Gonzalez-Aseguinolaza, G. Sano, M. Tsuji, and F. Zavala. 2001. Detection of malaria liver-stages in mice infected through the bite of a single Anopheles mosquito using a highly sensitive real-time PCR. Int. J. Parasitol. 31:1499-1502.
Carruthers, V. B. (1999). Armed and dangerous: Toxoplasma gondii uses an arsenal of secretory proteins to infect host cells. Parasitol Int 48, 1-10.
Carruthers, V. B., and Blackman, M. J. (2005). A new release on life: emerging concepts in proteolysis and parasite invasion. Mol Microbiol 55, 1617-1630.
Coppi, A., C. Pinzon-Ortiz, C. Hutter, and P. Sinnis. 2004. The Plasmodium circumsporozoite protein is proteolytically processed during cell invasion. J. Exp. Med. 201:27-33.
Coppi A., Cabinian M., Mirelman D. and Sinnis P. 2006 Antimalarial Activity of Allicin, a Biologically Active Compound From Garlic Cloves. Antimicrobial Agents and Chemotherapy 50:1731-1737.
Coppi, A., Tewari, R., Bishop, J., Lawrence, R., Esko, J., Billker, O., and Sinnis, P. (2007). Heparan Sulfate Proteoglycans Provide a Signal to Sporozoites to Stop Migrating and to Productively Invade Cells. Manuscript Submitted.
Eichinger, D. J., Arnot, D. E., Nussenzweig, V., and Enea, V. (1986). Circumsporozoite protein of Plasmodium berghei: gene cloning and identification of the immunodominant epitope. Mol Cell Biol 6, 3965-3972.
Eilat, S., Y. Oestraicher, A. Rabinkov, D. Ohad, D. Mirelman, A. Battler, M. Eldar, and Z. Vered. 1995. Alteration of lipid profile in hyperlipidemic rabbits by allicin, an active constituent of garlic. Coron. Artery Dis. 6:985-990.
Frevert, U., Sinnis, P., Cerami, C., Shreffler, W., Takacs, B., and Nussenzweig, V. (1993). Malaria circumsporozoite protein binds to heparan sulfate proteoglycans associated with the surface membrane of hepatocytes. J Exp Med 177, 1287-1298.
Gantt, S. M., Myung, J. M., Briones, M. R. S., Li, W. D., Corey, E. J., Omura, S., Nussenzweig, V., and Sinnis, P. (1998). Proteasome Inhibitors Block Development of Plasmodium spp. Antimicrob Ag Chem 42, 2731-2738.
Greenwood, B. M. (2005). Malaria. Lancet 365, 1487-1498.
Harlow, E., and D. Lane. 1988. Antibodies, a laboratory manual. Cold Spring Harbor Laboratories, Cold Spring Harbor.
Harris, L. C., S. L. Cottrel, S. Plummer, and e. al. 2001. Antimicrobial properties of Allium sativum (garlic). Applied Microbiol. Biotechnol. 57:282-286.
Hoffman, S. L., Goh, L. M. L., Luke, T. C., Schneider, I., Le, T. P., Doolan, D. L., Sacci, J., de la Vega, P., Dowler, M., Paul, C., et al. (2002). Protection of humans against malaria by immunization with radiation-attenuated Plasmodium falciparum sporozoites. J Inf Dis 185, 1155-1164.
Hollingdale, M. R., Nardin, E. H., Tharavanij, S., Schwartz, A. L., and Nussenzweig, R. S. (1984). Inhibition of entry of Plasmodium falciparum and P. vivax sporozoites into cultured cells; an in vitro assay of protective antibodies. J Immunol 132, 909-913.
Hollingdale, M. R., Zavala, F., Nussenzweig, R. S., and Nussenzweig, V. (1982). Antibodies to the protective antigen of Plasmodium berghei sporozoites prevent entry into cultured cells. J Immunol 128, 1929-1930.
Howell, S. A., C. Withers-Martinez, C. H. M. Kochen, A. W. Thomas, and M. J. Blackman. 2001. Proteolytic processing and primary structure of Plasmodium falciparum apical membrane antigen-1. J. Biol. Chem. 276:31311-31320.
Janse, C. J., Franke-Fayard, B., Mair, G. R., Ramesar, J., Thiel, C., Engelmann, S., Matuschewski, K., van Gemert, G. J., Sauerwein, R. W., and Waters, A. P. (2006). High efficiency transfection of Plasmodium berghei facilitates novel selection procedures. Mol Biochem Parasitol 145, 60-70.
Kappe, S. H., Buscaglia, C. A., Bergman, L. W., Coppens, I., and Nussenzweig, V. (2004). Apicomplexan gliding motility and host cell invasion: overhauling the motor model. Trends Parasitol 20, 13-16.
Kim, K. 2004. Role of proteases in host cell invasion by Toxoplasma gondii and other Apicomplexa. Acta Trop. 91:69-81.
Kumar, K. A., Oliveira, G. A., Edelman, R., Nardin, E., and Nussenzweig, V. (2004). Quantitative Plasmodium sporozoite neutralization assay. J Immunol Meth 292, 157-164.
Kumar, K. A., Sano, G., Boscardin, S., Nussenzweig, R. S., Nussenzweig, M. C., Zavala, F., and Nussenzweig, V. (2006). The circumsporozoite protein is an immunodominant protective antigen in irradiated sporozoites. Nature 444, 937-940.
Matsuoka, H., Yoshida, S., Hirai, M., and Ishii, A. (2002). A rodent malaria, Plasmodium berghei, is experimentally transmitted to mice by merely probing of infective mosquito, Anopheles stephensi. Parasitol Int 51, 17-23.
McKerrow, J. H. 1999. Cysteine protease inhibitors as chemotherapy for parasitic infections. Bioorganic Med. Chem. 7:639-644.
McKerrow, J. H. 1999. Development of cysteine protease inhibitors as chemotherapy for parasitic diseases: insights on safety, target validation, and mechanism of action. Inter. J. Parasitol. 29:833-837.
McKerrow, J. H., E. Sun, P. J. Rosenthal, and J. Bouvier. 1993. The proteases and pathogenicity of parasitic protozoa. Annu. Rev. Microbiol. 47:821-853.
Medica, D. L., and Sinnis, P. (2005). Quantitative Dynamics of Plasmodium yoelii Sporozoite Transmission by Infected Anopheline Mosquitoes Feeding on Vertebrate Hosts. Infect Immun 73, 4363-4369.
Menard, R. (2000). The journey of the malaria sporozoite through its hosts: two parasite proteins lead the way. Microb Inf 2, 633-642.
Menard, R., and Janse, C. (1997). Gene targeting in malaria parasites. Methods: A Companion to Methods in Enzymology 13, 148-157.
Menard, R., Sultan, A. A., Cortes, C., Altszuler, R., van Dijk, M. R., Janse, C. J., Waters, A. P., Nussenzweig, R. S., and Nussenzweig, V. (1997). Circumsporozoite protein is required for development of malaria sporozoites in mosquitoes. Nature 385, 336-340.
Mirelman, D., T. Miron, A. Rabinkov, and e. al. 1997. Immobilized alliinase and continuous production of allicin. PCT Publication No. WO 97/39115. U.S. Pat. No. 6,689,588.
Mirelman, D., D. Monheit, and S. Varon. 1987. Inhibition of growth of Entamoeba histolytica by allicin, the active principle of garlic (Allium sativum). J. Infect. Dis. 156:243-244.
Miron, T., A. Rabinkov, D. Mirelman, M. Wilchek, and L. Weiner. 2000. The mode of action of allicin: its ready permeability through phospholipid membranes may contribute to its biological activity. Biochem. et Biophys. Acta 1463:20-30.
Mo Myung, J., Marshall, P., and Sinnis, P. (2004). The Plasmodium circumsporozoite protein is involved in mosquito salivary gland invasion by sporozoites. Mol Biochem Parasitol 133, 53-59.
Mota, M., Pradel, G., Vanderberg, J. P., Hafalla, J. C. R., Frevert, U., Nussenzweig, R. S., Nussenzweig, V., and Rodriguez, A. (2001). Migration of Plasmodium sporozoites through cells before infection. Science 291, 141-144.
Nardin, E. H., Nussenzweig, V., Nussenzweig, R. S., Collins, W. E., Harinasuta, K. T., Tapchaisri, P., and Chomcharn, Y. (1982). Circumsporozoite proteins of human malaria parasites Plasmodium falciparum and Plasmodium vivax. J Exp Med 156, 20-30.
Perez, H. A., M. de la Rosa, and R. Apitz. 1994. In vivo activity of ajoene against rodent malaria. Antimicrob. Agents Chemother. 38:337-339.
Pinzon-Ortiz, C., J. Friedman, J. Esko, and P. Sinnis. 2001. The binding of the circumsporozoite protein to cell surface heparan sulfate proteoglycans is required for Plasmodium sporozoite attachment to target cells. J. Biol. Chem. 276:26784-26791.
Potocnjak, P., Yoshida, N., Nussenzweig, R. S., and Nussenzweig, V. (1980). Monovalent fragments (Fab) of monoclonal antibodies to a sporozoite surface antigen (Pb44) protect mice against malarial infection. J Exp Med 151, 1504-1513.
Rabinkov, A., T. Miron, L. Konstantinovski, M. Wilchek, D. Mirelman, and L. Weiner. 1998. The mode of action of allicin: trapping of radicals and interaction with thiol containing proteins. Biochim. Biophys. Acta 1379:233-244.
Rathore, D., Sacci, J. B., de la Vega, P., and McCutchan, T. F. (2002). Binding and invasion of liver cells by Plasmodium falciparum sporozoites. Essential involvement of the amino terminus of circumsporozoite protein. J Biol Chem 277, 7092-7098.
Renia, L., F. Miltgen, y. Charoenvit, T. Ponnudurai, J. P. Verhave, W. E. Collins, and D. Mazier. 1988. Malaria sporozoite penetration: A new approach by double staining. J. Immunol. Methods 112:201-205.
Reuter, H. D., H. P. Koch, and L. D. Lawson. 1996. Therapeutic effects and applications of garlic and its preparations., p. 135-213. In H. P.
Koch and L. D. Lawson (ed.), Garlic: the science and therapeutic application of Allium sativum L. and related species. Williams and Wilkins, Baltimore.
Rosenthal, P. J., P. S. Sijwali, A. Singh, and B. R. Shenai. 2002. Cysteine proteases of malaria parasites: Targets for chemotherapy. Curr. Pharmaceutical Design 8:1659-1672.
Shadkchan, Y., E. Shemesh, D. Mirelman, T. Miron, A. Rabinkov, M. Wilchek, and N. Osherov. 2004. Efficacy of allicin, the reactive molecule of garlic, in inhibiting Aspergillus spp. in vitro, and in a murine model of disseminated aspergillosis. J. Antimicrob. Chemother. 53:832-836.
Sidjanski, S. P., Vanderberg, J. P., and Sinnis, P. (1997). Anopheles stephensi salivary glands bear receptors for region I of the circumsporozoite protein of Plasmodium falciparum. Mol Biochem Parasitol 90, 33-41.
Sinnis, P., Clavijo, P., Fenyo, D., Chait, B., Cerami, C., and Nussenzweig, V. (1994). Structural and functional properties of region II-plus of the malaria circumsporozoite protein. J Exp Med 180, 297-306.
Sinnis, P., and Coppi, A. (2007). A Long and Winding Road: The Plasmodium Sporozoite's Journey in the Mammalian Host. Parasitol Int 56, 171-178.
Sinnis, P., and Nardin, E. (2002). Sporozoite Antigens: Biology and Immunology of the Circumsporozoite Protein and Thrombospondin Related Anonymous Protein., In Malaria Immunology, P. Perlmann, and M. Troye-Blomberg, eds. (Basel Switzerland: S. Karger Press), pp. 70-96.
Sultan, A. A., V. Thathy, U. Frevert, K. J. H. Robson, A. Crisanti, V. Nussenzweig, R. S. Nussenzweig, and R. Menard. 1997. TRAP is necessary for gliding motility and infectivity of Plasmodium sporozoites. Cell 90:511-522.
Tewari, R., Spaccapelo, R., Bistoni, F., Holder, A. A., and Crisanti, A. (2002). Function of region I and II adhesive motifs of Plasmodium falciparum circumsporozoite protein in sporozoite motility and infectivity. J Biol Chem 277, 47613-47618.
Thathy, V., Fujioka, H., Gantt, S., Nussenzweig, R., Nussenzweig, V., and Menard, R. (2002). Levels of circumsporozoite protein in the Plasmodium oocyst determine sporozoite morphology. EMBO J 21, 1586-1596.
Tsuji, M., Mattei, D., Nussenzweig, R. S., Eichinger, D., and Zavala, F. (1994). Demonstration of heat-shock protein 70 in the sporozoite stage of malaria parasites. Parasitol Res 80, 16-21.
Uchida, Y., T. Takahashi, and N. Sate. 1975. The characteristics of the antibacterial activity of garlic. Jpn. J. Antibiotics 28:638-642.
Vanderberg, J. P., and R. W. Gwadz. 1980. Malaria: Pathology, Vector Studies, and Culture, vol. 2. Academic Press, New York.
Wang, Q., Fujioka, H., and Nussenzweig, V. (2005). Exit of Plasmodium sporozoites from oocysts is an active process that involves the circumsporozoite protein. PLoS Pathog 1, e9.
Willis, E. 1956. Enzyme inhibition by allicin, the active principle of garlic. Biochem. J. 63:514-520.
Yoshida, N., R. S. Nussenzweig, P. Potocnjak, V. Nussenzweig, and M. Aikawa. 1980. Hybridoma produces protective antibodies directed against the sporozoite stage of Malaria parasite. Science 207:71-73.

What is claimed is:

1. A composition for preventing malaria infection comprising an inhibitor.

2. The composition according to claim 1, wherein said inhibitor is a steric inhibitor that targets a minor repeat region of circumsporozoite protein (CSP).

3. The composition according to claim 1, wherein said inhibitor is a direct inhibitor that targets region I of CSP.

4. The composition according to claim 1, wherein said inhibitor is an antibody.

5. The composition according to claim 4, wherein said antibody is selected from the group consisting of mAb 3D11, mAb 2A10, and mAb 2E6.

6. The composition according to claim 3, wherein said antibody is further defined as low titers of the antibody.

7. A pharmaceutical composition for preventing malaria infection comprising an effective amount of an inhibitor and a pharmaceutical carrier.

8. The pharmaceutical composition according to claim 7, wherein said inhibitor is a steric inhibitor that targets a minor repeat region of circumsporozoite protein (CSP).

9. The pharmaceutical composition according to claim 7, wherein said inhibitor is a direct inhibitor that targets region I.

10. The pharmaceutical composition according to claim 7, wherein said inhibitor is an antibody.

11. The pharmaceutical composition according to claim 10, wherein said antibody is selected from the group consisting of mAb 3D11, mAb 2A10, and mAb 2E6.

12. The pharmaceutical composition according to claim 10, wherein said antibody is further defined as low titers of the antibody.

13. A method of malaria infection prophylaxis including the step of:
administering an effective amount of the composition according to claim 1.

14. The method of claim 13, further including the step of targeting a site chosen from the group consisting of a minor repeat region and region I of circumsporozoite protein (CSP) with the composition of claim 1.

15. The method of claim 13, wherein said administering step is further defined as administering low titers of an antibody.

16. A method of malaria prophylaxis including the step of inhibiting an interaction between a protease of a sporozoite and circumsporozoite protein.

17. The method of claim 16, wherein the inhibiting step is chosen from the group consisting of directly inhibiting region I and sterically inhibiting a minor repeat region.

18. A method of preventing sporozoite cell invasion including the step of administering an effective amount of the composition according to claim 1.

19. A method of preventing circumsporozoite processing including the step of administering an effective amount of the composition according to claim 1.

20. A method of preventing malaria infection including the step of preventing sporozoite cell invasion of a host cell by inhibiting circumsporozoite protein processing.

21. The method according to claim 20, wherein said inhibiting step includes inhibiting cleavage of the sporozoite's circumsporozoite protein by an inhibitor.

22. The method of claim 21, wherein the inhibitor is a steric inhibitor that targets a minor repeat region of circumsporozoite protein (CSP).

23. The method of claim 21, wherein the inhibitor is a direct inhibitor that targets region I of CSP.

24. The method of claim 21, wherein the inhibitor is an antibody.
